SEQUENCE LISTING (2 sequences)

SEQ ID NO: 1 - protein, 9 aa, synthetic construct
CRGDKGPDC

SEQ ID NO: 2 - protein, 8 aa, synthetic construct
RGDKGPDC

(19) United States
(12) Patent Application Publication
(10) Pub. No.: US 2026/0325669 A1
(43) Pub. Date: Oct. 22, 2026

(54) EXOSOME COMPOSITIONS FOR TARGETING CANCER CELLS AND ASSOCIATED METHODS

(71) Applicant: University of Central Florida Research Foundation, Inc., Orlando, FL (US)

(72) Inventors: Dinender Singla, Orlando, FL (US); Chandrakala Aluganti Narasimhulu, Orlando, FL (US)

(21) Appl. No.: 19/631,883

(22) Filed: Mar. 27, 2026

Related U.S. Application Data
(60) Provisional application No. 63/779,409, filed on Mar. 28, 2025.

(51) Int. Cl. A61K 47/69; A61K 31/198; A61K 31/282; A61K 31/4184; A61K 31/475; A61K 31/513; A61K 31/519; A61K 31/675; A61K 31/7048; A61K 31/7068; A61K 33/243; A61K 47/60; A61K 47/62; A61P 35/00

(52) U.S. Cl. CPC A61K 47/6911; A61K 31/198; A61K 31/282; A61K 31/4184; A61K 31/475; A61K 31/513; A61K 31/519; A61K 31/675; A61K 31/7048; A61K 31/7068; A61K 33/243; A61K 47/60; A61K 47/62; A61P 35/00

(57) ABSTRACT

The present technology includes compositions comprising exosomes (i.e., “exosome compositions”) having one or more tumor-specific peptides (TSPs) (TSP-Exos) that guide the exosome composition to a desired target. The TSPs of the present technology may (i) bind to at least a portion of a cancer cell (e.g., a tumor cell) and/or a tumor microenvironment, or (ii) otherwise colocalize one or more exosomes with the cancer cell and/or the tumor microenvironment. The exosome compositions may penetrate the tumor microenvironment and are at least partially internalized by the cancer cell upon binding to at least a portion of the cancer cell by the TSP. For example, an exosome or a cargo thereof (e.g., an anticancer agent) is internalized by the cancer cell upon binding of the TSP.

### CROSS-REFERENCE TO RELATED APPLICATION

This application claims the benefit of U.S. Provisional Patent Application No. 63/779,409, filed Mar. 28, 2025, the entirety of which is incorporated herein by reference.

### INCORPORATION BY REFERENCE OF SEQUENCE LISTING

This application contains an ST.26 compliant Sequence Listing, which is submitted concurrently in xml format and hereby incorporated by reference in its entirety. The .xml copy, created on Mar. 27, 2026, is titled “269A-419857-WO.xml,” and is 2,690 bytes in size.

### BACKGROUND

Cancer remains a leading cause of death worldwide and a significant public health concern. Triple-negative breast cancer (TNBC) is a distinct subtype, representing approximately 10-15% of breast cancers, and is generally more aggressive than other forms. TNBC lacks estrogen receptors, progesterone receptors, and human epidermal growth factor receptor 2 (HER2), rendering many established hormone- and HER2-targeted therapies ineffective. TNBC tumors are also heterogeneous and often metastasize rapidly, and features of the tumor microenvironment may hinder therapeutic recognition and penetration.

Doxorubicin (DOX) is widely used against a range of cancers, including TNBC, yet its clinical utility is limited by non-specific distribution, injury to healthy tissue, short effective exposure, significant adverse effects (notably cardiotoxicity), and the emergence of resistance. Reported resistance mechanisms include inadequate drug penetration into the tumor microenvironment, reducing effective intracellular interaction with nucleic acids and diminishing cytotoxicity. Additionally, as cancer survival improves, treatment-associated cardiac toxicity has become an increasing clinical burden. Accordingly, there is a need for technologies that enhance tumor-selective delivery and efficacy of chemotherapeutics, such as DOX, while minimizing off-target exposure.

### SUMMARY

The present technology comprises exosome compositions comprising or consisting of an exosome at least partially conjugated to one or more tumor-specific peptides (TSPs) that bind at least a portion of a cancer cell or a tumor microenvironment.

In some embodiments, the exosome compositions further comprise one or more anticancer agents at least partially encapsulated within the exosome.

In some embodiments, the exosome compositions further comprise one or more anticancer agents at least partially encapsulated within the exosome.

In some embodiments, the present technology includes an exosome composition comprising or consisting of (i) an exosome at least partially conjugated to one or more TSPs that bind at least a portion of a cancer cell or a tumor microenvironment, and (ii) one or more anticancer agents at least partially encapsulated within the exosome.

In some embodiments, the present technology comprises a method of targeting a cancer in a subject in need thereof relative to a control, the method comprising administering to the subject:
an exosome composition comprising or consisting of (i) an exosome at least partially conjugated to one or more TSPs that bind at least a portion of a cancer cell or a tumor microenvironment, and (ii) one or more anticancer agents at least partially encapsulated within the exosome.

In some embodiments, the cancer is triple negative breast cancer (TNBC).

In some embodiments, the cancer is selected from the group consisting of breast cancer, lung cancer, colorectal cancer, prostate cancer, pancreatic cancer, ovarian cancer, cervical cancer, endometrial cancer, liver cancer, gastric cancer, esophageal cancer, kidney cancer, bladder cancer, melanoma, thyroid cancer, head and neck cancer, brain cancer, testicular cancer, sarcoma, bone cancer, lymphoma, multiple myeloma, and leukemia.

In some embodiments, the present technology comprises a method of enhancing one or more anticancer agent effects in a subject in need thereof, relative to a control, the method comprising administering to the subject:
an exosome composition comprising or consisting of (i) an exosome at least partially conjugated to one or more TSPs that bind at least a portion of a cancer cell or a tumor microenvironment, and (ii) one or more anticancer agents at least partially encapsulated within the exosome.

In some embodiments, the one or more anticancer agent effects is selecting from the group consisting of reducing or preventing an increase in a tumor size, reducing or preventing an increase in a tumor volume, and/or reducing or preventing a cancer metastasis level in a subject in need thereof, relative to a control.

In some embodiments, the method of reducing or preventing an anticancer agent toxicity level in a subject in need thereof, relative to a control, the method comprising administering to the subject:
an exosome composition comprising or consisting of (i) an exosome at least partially conjugated to one or more TSPs that bind at least a portion of a cancer cell or a tumor microenvironment, and (ii) one or more anticancer agents at least partially encapsulated within the exosome.

In some embodiments, the anticancer agent toxicity comprises anticancer agent-induced cardiomyopathy or cell death in non-cancer cell populations.

In some embodiments, the anticancer agent-induced cardiomyopathy is doxorubicin (DOX)-induced cardiomyopathy.

In some embodiments, the method reduces one or more of a cardiac fibrosis level, a cytoplasmic vacuolization level, or a cardiac apoptosis level, relative to a control.

In some embodiments, the one or more anticancer agents is selected from the group consisting of an alkylating agent, a platinum agent, an antimetabolite, a topoisomerase inhibitor, a microtubule-targeting agent, and a cytotoxic agent.

In some embodiments, the alkylating agent is selected from the group consisting of cyclophosphamide, ifosfamide, melphalan, and bendamustine.

In some embodiments, the platinum agent is selected from the group consisting of cisplatin, carboplatin, and oxaliplatin.

In some embodiments, the antimetabolite is selected from the group consisting of 5-fluorouracil (5-FU), capecitabine, methotrexate, cytarabine, gemcitabine, and pemetrexed.

In some embodiments, the topoisomerase inhibitor is selected from the group consisting of doxorubicin (DOX), epirubicin, irinotecan, topotecan, etoposide, and teniposide.

In some embodiments, the microtubule-targeting agent is selected from the group consisting of paclitaxel, docetaxel, vincristine, vinblastine, vinorelbine, and cabazitaxel.

In some embodiments, the cytotoxic agent is selected from the group consisting of bleomycin, dacarbazine, and temozolomide.

In some embodiments, the exosome composition is at least partially internalized by the cancer cell.

In some embodiments, the portion of the cancer cell or the tumor microenvironment comprises a cell surface protein.

In some embodiments, the cell surface protein is a transmembrane protein.

In some embodiments, the transmembrane protein is an integrin or a subunit thereof.

In some embodiments, the integrin is selected from the group consisting of an arginine-glycine-aspartic acid (RGD)-binding integrin, a collagen-binding integrin, a laminin-binding integrin, and a leukocyte-specific integrin.

In some embodiments, the one or more TSPs comprise an RGD motif.

In some embodiments, the one or more TSPs comprise or consist of an amino acid sequence at least about 100%, 99%, 98%, 95%, 90%, 85%, 80%, 75%, 70%, 65%, 60%, or 50% identical to the amino acid sequence of CRGDKGPDC (SEQ ID NO: 1).

In some embodiments, the present technology comprises an exosome composition comprising or consisting of (i) an exosome at least partially conjugated to one or more TSPs comprising or consisting of an amino acid sequence of CRGDKGPDC (SEQ ID NO: 1) that at least partially bind a cell surface integrin of a cancer cell, and (ii) DOX at least partially encapsulated within the exosome.

In some embodiments, the integrin is selected from the group consisting of αvβ1, αvβ3, αvβ5, αvβ6, αvβ8, α8β1, αIIbβ3, α1β1, α2β1, α3β1, α10β1, α11β1, α6β1, α7β1, α6β4, αLβ2, αMβ2, αXβ2, αDβ2, α4β1, α4β7, α9β1, and αEβ7.

In some embodiments, the exosome is at least partially conjugated to the one or more TSPs by a lipid-polymer conjugate.

In some embodiments, the lipid-polymer conjugate comprises or consists of one or more of a phospholipid, a polymer, and a functional group.

In some embodiments, (i) the phospholipid is 1,2-distearoyl-sn-glycero-3-phosphoethanolamine (DSPE); (ii) the polymer is poly(ethylene glycol) (PEG5000); and/or (iii) the functional group is an azide functional group.

In some embodiments, the exosome is a mesenchymal stem cell-derived exosomes (MSc-exosome).

In some embodiments, the exosome is selected from the group consisting of an adipose-derived stem cell exosome (asc-exosome), a bone marrow-derived mesenchymal stromal/stem cell exosome (bmsc-exosome), an umbilical cord mesenchymal stromal/stem cell exosome (ucmsc-exosome), a dendritic cell-derived exosome (dc-exosome), a T cell-derived exosome, a natural killer cell-derived exosome (NK cell-derived exosome), a B cell-derived exosome, a macrophage-derived exosome, an M1 macrophage-derived exosome (M1-exosome), an M2 macrophage-derived exosome (m2-exosome), a platelet-derived exosome, an erythrocyte-derived exosome, an endothelial cell-derived exosome, a fibroblast-derived exosome, a neural stem cell-derived exosome, a neuron-derived exosome, an astrocyte-derived exosome, a microglia-derived exosome, a cardiomyocyte-derived exosome, a hepatocyte-derived exosome, a tumor-derived exosome (tdex), a cancer-associated fibroblast-derived exosome (caf-derived exosome), a serum-derived exosome, a plasma-derived exosome, a urine-derived exosome, a saliva-derived exosome, a breast milk-derived exosome, an ascites-derived exosome, and a cerebrospinal fluid-derived exosome (csf-derived exosome).

In some embodiments, the exosome comprises a particle size of about 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, or 200 nm.

In some embodiments, the exosome comprises a zeta potential of about −100 mV, −90 mV, −80 mV, −75 mV, −70 mV, −60 mV, −65 mV, −50 mV, −45 mV, −40 mV, −35 mV, −30 mV, −20 mV, or −15 mV.

In some embodiments, the exosome compositions comprise an exosome:TSP weight ratio of at least about 1:2500, 1:3000, 1:4000, 1:5000, 1:5500, 1:6000, 1:6500, 1:7000, 1:8000, 1:9000, or 1:10000.

In some embodiments, the one or more anticancer agents are loaded into the exosome compositions with a weight ratio of TSP and exosomes:anticancer agent of about 10:1, 11:1, 12:1, 13:1, 14:1, 15:1, 16:1, 17:1, 18:1, 19:1, or 20:1.

In some embodiments, the one or more anticancer agents comprise or consist of two or more anticancer agents.

In some embodiments, the DOX is loaded into the exosome compositions at a weight ratio of TSP and exosomes:DOX of about 14:1.

In some embodiments, the cancer cell is selected from the group consisting of a breast cancer cell, a lung cancer cell, a colorectal cancer cell, a prostate cancer cell, a pancreatic cancer cell, an ovarian cancer cell, a cervical cancer cell, an endometrial cancer cell, a liver cancer cell, a gastric cancer cell, an esophageal cancer cell, a kidney cancer cell, a bladder cancer cell, a melanoma cell, a thyroid cancer cell, a head and neck cancer cell, a brain cancer cell, a testicular cancer cell, a sarcoma cell, a bone cancer cell, a lymphoma cell, a multiple myeloma cell, and a leukemia cell.

In some embodiments, the cancer cell is a triple negative breast cancer (TNBC) cell.

In some embodiments, the subject has or is at risk of developing TNBC.

In some embodiments, the subject has or is at risk of developing primary breast cancer or metastatic breast cancer.

In some embodiments, the subject has received or is receiving an anticancer agent or a cancer treatment.

In some embodiments, the subject has not received or an anticancer agent or a cancer treatment.

In some embodiments, the cancer treatment is selected from the group consisting of an immunotherapy, a radiation therapy, and a surgical therapy.

In some embodiments, the exosome composition is formulated in a pharmaceutical composition.

In some embodiments, the pharmaceutical composition further comprises one or more pharmaceutically acceptable carriers or excipients.

In some embodiments, the pharmaceutical composition is formulated for oral, rectal, vaginal, ocular, intranasal, topical, parenteral, injectable delivery, or from a surgical device.

In some embodiments, the pharmaceutical composition is administered to the subject orally, rectally, vaginally, ocularly, intranasally, topically, parenterally, by injection, or form a surgical device.

In some embodiments, the injectable delivery comprises intravenous (IV), subcutaneous (SC), intramuscular (IM), intraperitoneal (IP), or intracerebroventricular (ICV) injection.

In some embodiments, the exosome composition is administered to the subject in an amount of about 1.5 mg/kg/day to about 45 mg/kg/day.

In some embodiments, the exosome composition is administered to the subject in an amount of about 0.01 mg/kg/day to about 10.0 mg/kg/day, of the one or more anticancer agents.

### BRIEF DESCRIPTION OF THE DRAWINGS

FIGS. 1A-1D illustrate characterization of exosomes derived from mesenchymal stem cells (mMScs) (mMSc-Exos), in accordance with the embodiments of the present technology. FIG. 1A illustrates particle size and distribution (30-150 nm in diameter) by Dynamic Light Scattering (DLS). FIG. 1B illustrates a representative image of mMSc-Exos FIG. 1A. FIG. 1C illustrates Zeta Potential measurements (negative charge) of mMSc-Exos of FIG. 1A. FIG. 1D illustrates presence of exosome protein biomarkers, Cluster of Differentiation 63 (CD63) and Heat Shock Protein 70 (HSP70). Scale bar: 100 nm. SEM: Scanning Electron Microscope.

FIGS. 2A-2C illustrates murine triple negative breast cancer (TNBC) cell uptake of mMSc-Exos having tumor-specific peptide (TSP) (TSP-mMSc-Exos), in accordance with the embodiments of the present technology. FIG. 2A shows a representative image of 1,1′-Dioctadecyl-3,3,3′,3′-Tetramethylindocarbocyanine (Dil) labeled TSP-mMS-Exos binding to and entering the TNBC cells. FIG. 2B shows a representative photomicrograph of TSP-mMSc-Exos uptake by the TNBC cells of FIG. 2A at different time points. FIG. 2C illustrates quantification of fluorescence intensity representing cellular uptake of FIGS. 2A and 2B. Scale bar: 100 μm. *p<0.05; **p<0.01; ***p<0.001; ****p<0.0001.

FIGS. 3A and 3B illustrate Dil-labeled TSP-mMSc-Exos binding to and entering human TNBC cells. FIG. 3A illustrates exosome uptake by the human TNBC cells at different time points. FIG. 3B illustrates quantification of fluorescence intensity for the exosome uptake of FIG. 3A. Scale bar: 100 μm; *p<0.05; **p<0.01; ***p<0.001; TSP: Tumor specific peptide.

FIGS. 4A and 4B illustrate doxorubicin (DOX) loading efficiency in TSP-mMSc-Exos in accordance with the embodiments of the present technology. FIG. 4A illustrates DOX loading efficiency (μg/mg) into TSP-mMSc-Exos. FIG. 4B illustrates DOX encapsulation efficiency into TSP-mMSc-Exos.

FIGS. 5A-5D illustrate TSP-mMSc-Exos loaded with DOX (TSP-DOX-Exos) enhance TNBC cell death after 24 hrs. FIG. 5A provides representative photomicrographs of TSP-DOX-Exos treated TNBC cells compared to TNBC cells treated with DOX alone. Arrows depict an increase in staining fluorescence. FIGS. 5B and 5C illustrate cell viability (as a percentage of control cells) and cell death (as a percentage of total cells), respectively. FIG. 5D illustrate an increase in TNBC cell apoptosis in TSP-DOX-Exos, compared to controls. Scale bar: 100 μm. ***p<0.001; ****p<0.0001. TSP: Tumor-specific peptide.

FIGS. 6A-6E illustrate breast tumor measurements in mice under various conditions (no tumor: Con; tumor without treatment: Tumor; tumor with DOX treatment: Tumor+DOX). FIGS. 6A-6C illustrate no significant reduction in breast tumor size and volume compared with the tumor group in mice treated with DOX. FIGS. 6D and 6E illustrate quantification of tumor size and tumor volume for the mice of FIGS. 6A-6C. respectively. n=7 animals/group. ***p<0.001; ****p<0.0001, ns: non-significant.

FIGS. 7A-7G illustrate identification of injected TSP-DOX-Exos in TNBC tumors in mice. Representative images depict (FIG. 7A) localization of 1,1′-Dioctadecyl-3,3,3′,3′-Tetramethylindotricarbocyanine lodide (DiR) labeled TSP-DOX-Exos in TNBC tumors in mice but not in (FIG. 7B) hearts. FIG. 7C illustrates DiR labeled TSP-DOX-Exos penetration in TNBC tumor slices but not in (FIG. 7D) heart slices. FIG. 7E illustrates TNBC tumor size reduction with TSP-DOX-Exos. FIGS. 7F and 7G illustrate representative photomicrographs showing that TSP-DOX-Exos inhibit tumor cell proliferation and induces apoptosis, respectively. Scale bar: 100 μm

FIGS. 8A-8D illustrates DOX-induced cardiotoxicity in tumor-treated mice. FIG. 8A illustrates representative photomicrographs displaying apoptosis (arrows). FIG. 8B illustrates apoptosis staining quantification for the cells of FIG. 8A. FIGS. 8C and 8D illustrate cardiac function for the mice of FIG. 8A as fractional shortening (FS, FIG. 8C) and ejection fraction (EF, FIG. 8D). n=6 animals/group. Scale bar: 100 μm. **p<0.01; ***p<0.001; ****p<0.0001.

FIGS. 9A-9FI illustrate TNBC tumor reduction in TSP-DOX-Exo treated mice. FIGS. 9A-9C show comparisons of tumors in untreated mice (tumor mice), mice treated with DOX alone (tumor+ DOX) and mice treated with the TSP-DOX-Exos of the present technology (Tumor+TSP-Dox-Exos), respectively. FIG. 9D showed quantified changes in tumor volume in these mice through day 37. FIGS. 9E and 9F show changes in tumor size and tumor volume between groups at the end of the study, respectively. n=4-5 animals/group. *p<0.05; **p<0.01.

FIGS. 10A and 10B illustrate TSP-DOX-Exos of the present technology reducing tumor growth in mice relative to untreated mice or mice treated with DOX alone. FIG. 10A shows a representative photomicrograph of TSP-DOX-Exos inhibiting tumor cell proliferation when comparing the murine groups of FIGS. 9A-9F. FIG. 10B shows quantified changes in tumor cell growth between these groups. Scale bar: 100 μm; n=5-6 animals/group. *p<0.05; ***p<0.001; ****p<0.0001.

FIGS. 11A and 11B illustrate TSP-DOX-Exos of the present technology increasing tumor cell death in mice relative to untreated mice or mice treated with DOX alone. FIG. 11A shows a representative photomicrograph of TSP-DOX-Exos inducing apoptosis when comparing the murine groups of FIGS. 9A-9F. FIG. 11B shows quantified changes in tumor cell death between these groups. Scale bar: 100 μm; n=5-7 animals/group. ****p<0.0001.

FIGS. 12A-12C illustrate TSP-DOX-Exos of the present technology enhancing TNBC cell death, compared to untreated control (Con), cells treated with DOX alone (DOX), and TSP-Exos lacking DOX (Dil-TSP-Exos). FIG. 12A is a representative photomicrograph depicting an increase in cell death in TSP-DOX-Exo treated cells compared to other assessed groups (arrows). FIGS. 12B and 12C show reduced TNBC cell viability and an increase in TNBC cell death with TSP-DOX-Exos treatment compared to other assessed groups of FIG. 12A, respectively. Scale bar: 100 μm; ***p<0.001; ****p<0.0001.

FIGS. 13A-13C show dose-dependent killing of human TNBC cells in culture with TSP-DOX-Exos treatment. FIG. 13A shows representative photomicrographs depicting an increase in TNBC cell death with increasing concentrations of TSP-DOX-Exo treatment compared to untreated controls (Con). FIGS. 13B and 13C show quantified changes in TNBC cell viability (FIG. 13B) and death (FIG. 13C) in the groups of FIG. 13A. Scale bar: 100 μm; n=8; *p<0.05; **p<0.01; ***p<0.001; ****p<0.0001.

### DETAILED DESCRIPTION

The present technology comprises exosome compositions for targeting cancer and associated methods. The exosome compositions may be used to deliver anticancer drugs to specific cancers by selectively binding to cancer cells and penetrating tumor microenvironments. Binding to cancer cells may include binding of the exosome composition or at least a portion thereof to a cancer cell antigen, such as a surface protein or peptide thereof of the cancer cell.

The following description is merely exemplary in nature and is not intended to limit the present technology, its applications, or its uses. It should be understood that throughout the drawings, corresponding reference numerals indicate like or corresponding parts and features. The description of specific examples indicated in various embodiments of the present technology are intended for purposes of illustration only and are not intended to limit the scope of the present technology disclosed herein. Moreover, recitation of multiple embodiments having stated features is not intended to exclude other embodiments having additional features or other embodiments incorporating different combinations of the stated features.

Furthermore, the detailed description of various embodiments herein makes reference to the accompanying drawing/FIGS, which show various embodiments by way of illustration. While the embodiments are described in sufficient detail to enable those skilled in the art to practice the present technology, it should be understood that other embodiments may be realized, and that logical and mechanical changes may be made without departing from the spirit and scope of the present technology. Thus, the detailed description herein is presented for purposes of illustration only and not of limitation. For example, steps or functions recited in any description, method, system, or process, may be executed in any order and are not limited to the order presented. Moreover, any of the steps or functions thereof may be outsourced to or performed by one or more third parties.

### DEFINITIONS

Unless defined otherwise, all technical and scientific terms used herein have the same meaning as commonly understood by those of ordinary skill in the art to which the present technology belongs. For the purposes of the present technology, the following terms are defined below.

The articles “a” and “an” are used herein to refer to one or to more than one (i.e., to at least one) of the grammatical object of the article. By way of example, “an element” means one element or more than one element. Likewise, any reference to singular includes plural embodiments, and any reference to more than one component may include a singular embodiment.

The term “about” means a quantity, level, value, number, frequency, percentage, dimension, size, amount, weight or length that varies by acceptable levels in the art. Typically, such variation may be as much 10% above and below a reference quantity, level, value, number, frequency, percentage, dimension, size, amount, weight or length and such variation may be influenced by standard applicable measurement practices. When the term “about” is used in conjunction with a numerical range, it modifies that range by extending the boundaries above and below the numerical values set forth.

The present invention may “comprise” (open ended) the components of the present invention (e.g., chemotherapy drugs) as well as other ingredients or elements described herein. As used herein, “comprising” means the elements recited, or their equivalent in structure or function, plus any other element or elements which are not recited. The terms “having” and “including” are also to be construed as open ended unless the context suggests otherwise.

Where used herein, the term “and/or” when used in a list of two or more items means that any one of the listed characteristics may be present, or any combination of two or more of the listed characteristics may be present. For example, if a composition of the instant invention is described as containing characteristics A, B, and/or C, the exosome composition may contain A feature alone; B alone; C alone; A and B in combination; A and C in combination; B and C in combination; or A, B, and C in combination.

As used herein, the term “subject” or “patient” refers to a mammal, including a primate, and, in some embodiments, a human, who has or is at risk of developing a cancer, such as breast cancer, including triple negative breast cancer (TNBC).

The terms “treat”, “treatment”, and “treating” may also refer to the reduction or inhibition of the progression and/or duration of a disease (e.g., cancer), the reduction or amelioration of the severity of the disease, and/or the amelioration of one or more symptoms thereof resulting from the administration of one or more therapies (e.g., an exosome composition of the present technology).

As used herein, a “composition” or a “pharmaceutical composition” refers to a mixture of the active ingredient with other chemical components, such as pharmaceutically acceptable carriers and/or excipients.

As used herein, a “pharmaceutically acceptable carrier” of the pharmaceutical composition refers to a carrier or diluent that does not cause significant irritation to an organism, does not abrogate the biological activity and properties of the administered active ingredient, and/or does not interact in a deleterious manner with the other components of the composition in which it is contained. The term “carrier” encompasses any excipient, binder, diluent, filler, salt, buffer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia Pa., 2005, which is incorporated herein by reference in its entirety). Some examples of physiologically acceptable carriers include antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.). An “excipient” of the first or the pharmaceutical composition refers to an inert substance added to a composition to further facilitate administration of a compound. Examples, without limitation, of excipients include calcium carbonate, calcium phosphate, various sugars and types of starch, cellulose derivatives, gelatin, vegetable oils, and polyethylene glycols.

The terms “administering” or “administer” include delivery of therapies (e.g., exosome compositions of the present technology or pharmaceutical compositions comprising the same) to a subject either by local or systemic administration.

As used herein, the terms “effective amount” or “therapeutically effective amount,” refer to that amount of the active ingredient being administered which will relieve to some extent one or more of the symptoms of the condition, disease, or disorder being treated.

As used herein, the term “dosage form” refers to a pharmaceutical composition in which a specific mixture (i.e., composition) of active ingredients (e.g., anticancer drug) and inactive components (excipients) are formulated in a particular shape or form to facilitated administration and accurate delivery of active ingredients, and/or to be presented in the market. Solid dosage forms include powder, granules, capsules, tablets/pills, cachets, troches, lozenges, gummies, suppositories. Optionally, a tablet dosage form may be fast dissolving, extended release (XR) or long-acting (LA), sustained release (SR), controlled release (CR), delayed release (DR), or enteric coating formulation. Semi-solid dosage forms include ointment, creams, paste, gels, poultices. Liquid dosage forms include solutions, syrups, collodions, droughts, elixirs, emulsions, suspension, enemas, gargles, linctuses, lotion, liniments, mouth washes, nasal drop, and paints. Gaseous dosage forms include aerosols, inhalations, and sprays. Examples of suitable pharmaceutical excipients for each dosage form are described in “Remington's Pharmaceutical Sciences.”

As used herein, the term “anticancer drug” refers to a cytotoxic or cytostatic agent to kill cancer cells, which also kills some normal tissue, targeting specific mutated genes or proteins.

The term “control” refers to any composition or subject used as a basis for comparison. A control subject includes, but is not limited to, an untreated subject, a subject treated with an anticancer agent alone, or a subject treated with a composition lacking one or more features of the present technology. The control may also comprise a composition having an exosome lacking one or more of a tumor specific peptide (TSP) or an anticancer agent of the present technology.

The terms “subject” refers to anyone being evaluated for disease, disorder, or condition or being administered an exosome composition or a pharmaceutical composition comprising the same. This includes people with or without diagnosed or confirmed disease or condition (e.g., cancer).

While the present technology is capable of being embodied in various forms, the description below of several embodiments is made with the understanding that the present technology is to be considered as an exemplification of the technology and is not intended to limit the present technology to the specific embodiments illustrated. Headings are provided for convenience only and are not to be construed to limit the present technology in any manner. Embodiments illustrated under any heading may be combined with embodiments illustrated under any other heading.

### Exosome Compositions

The present technology includes compositions comprising exosomes (i.e., “exosome compositions”) having one or more tumor-specific peptides (TSPs) (TSP-Exos) that guide the exosome composition to a desired target, such as a cancer cell. The TSPs of the present technology may (i) bind to at least a portion of the cancer cell (e.g., a tumor cell) and/or a tumor microenvironment, or (ii) otherwise colocalize one or more exosomes with the cancer cell and/or the tumor microenvironment. The exosome compositions may penetrate the tumor microenvironment and are at least partially internalized by the cancer cell upon binding to at least a portion of the cancer cell by the TSP. For example, an exosome or a cargo thereof (e.g., an anticancer agent) may be internalized by the cancer cell upon binding of the TSP.

In some embodiments, the portion of the cancer cell comprises or consists of a cancer antigen. In some embodiments, the portion of the cancer cell comprises or consists of two or more amino acids of a protein or a peptide thereof of the cancer cell or the tumor microenvironment. In some embodiments, the portion of the cancer cell comprises an integrin.

Nonlimiting examples of cancer cells include breast cancer cells, lung cancer cells, colorectal cancer cells, prostate cancer cells, pancreatic cancer cells, ovarian cancer cells, cervical cancer cells, endometrial cancer cells, liver cancer cells, gastric cancer cells, esophageal cancer cells, kidney cancer cells, bladder cancer cells, melanoma cells, thyroid cancer cells, head and neck cancer cells, brain cancer cells, testicular cancer cells, sarcoma cells, bone cancer cells, lymphoma cells, multiple myeloma cells, and leukemia cells. In some embodiments, the breast cancer cell is a triple negative breast cancer (TNBC) cells.

### Exosomes

The exosomes of the present technology may be derived from various cell origins or biofluid sources. Nonlimiting examples of exosomes of the present technology include mesenchymal stem cell-derived exosomes (MSc-exosomes), adipose-derived stem cell exosomes (asc-exosomes), bone marrow-derived mesenchymal stromal/stem cell exosomes (bmsc-exosomes), umbilical cord mesenchymal stromal/stem cell exosomes (ucmsc-exosomes), dendritic cell-derived exosomes (dc-exosomes), T cell-derived exosomes, natural killer cell-derived exosomes (NK cell-derived exosomes), B cell-derived exosomes, macrophage-derived exosomes, M1 macrophage-derived exosomes (M1-exosomes), M2 macrophage-derived exosomes (m2-exosomes), platelet-derived exosomes, erythrocyte-derived exosomes, endothelial cell-derived exosomes, fibroblast-derived exosomes, neural stem cell-derived exosomes, neuron-derived exosomes, astrocyte-derived exosomes, microglia-derived exosomes, cardiomyocyte-derived exosomes, hepatocyte-derived exosomes, tumor-derived exosomes (tdex), cancer-associated fibroblast-derived exosomes (caf-derived exosomes), serum-derived exosomes, plasma-derived exosomes, urine-derived exosomes, saliva-derived exosomes, breast milk-derived exosomes, ascites-derived exosomes, and cerebrospinal fluid-derived exosomes (csf-derived exosomes).

In some embodiments, the exosome is derived from a human or a non-human tissue. The non-human tissue may be murine. In some embodiments, the murine-derived exosome is a murine MSc-exosome (mMSc-exosome).

In some embodiments, the exosomes of the present technology comprise a particle size of about 20 nm to about 200 nm. In some embodiments, the particle size is a mean particle size of two or more exosomes.

In some embodiments, the particle size is about 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, or 200 nm.

In some embodiments, the particle size is at least 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, or 200 nm.

In some embodiments, the particle size is at least about 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, or 200 nm.

The exosomes of the present technology may comprise a zeta potential of about −100 mV to about −15 mV. In some embodiments, the zeta potential is a mean zeta potential of two or more exosomes.

In some embodiments, the zeta potential is about −100 mV, −90 mV, −80 mV, −75 mV, −70 mV, −60 mV, −65 mV, −50 mV, −45 mV, −40 mV, −35 mV, −30 mV, −20 mV, or −15 mV.

In some embodiments, the zeta potential is at least-100 mV, −90 mV, −80 mV, −75 mV, −70 mV, −60 mV, −65 mV, −50 mV, −45 mV, −40 mV, −35 mV, −30 mV, −20 mV, or −15 mV.

In some embodiments, the zeta potential is at least about −100 mV, −90 mV, −80 mV, −75 mV, −70 mV, −60 mV, −65 mV, −50 mV, −45 mV, −40 mV, −35 mV, −30 mV, −20 mV, or −15 mV.

The exosomes of the present technology may comprise one or more exosomal markers, including presence of CD63 (NCBI Accession(s): NP_001400212.1, NP_001244321.1, NP_001400215.1, NP_001400216.1, EAW96828.1, AAH13017.1), heat shock protein 70 (HSP70) (NCBI Accession(s): NP_005336.3; NP_005337.2), CD9 (NCBI Accession: NP_001760.1), CD8 (NCBI Accession(s): NP_001759.1, NP_004922.1), tumor susceptibility gene 101 protein (TSG101) (NCBI Accession: NP_006283.1). The exosomal marker may be present in an amount that is 20% to 1000% or more, relative to a control.

The exosomes of the present technology may be labeled for identification of any means known in the art. This includes, but is not limited to, a fluorescent dye. Nonlimiting examples of fluorescent dyes include 1,1′-dioctadecyl-3,3,3′,3′-tetramethylindocarbocyanine perchlorate (Dil) and 1,1′-dioctadecyl-3,3,3′,3′-tetramethylindotricarbocyanine iodide (DiR).

### Tumor-Specific Peptides (TSPs)

The exosomes of the present technology may be at least partially conjugated to one or more TSPs. The TSPs are utilized to guide or otherwise localize the exosome compositions to a cancer cell and/or tumor microenvironment through binding of the TSP to a desired target.

The TSPs may be conjugated to the exosome to generate exosome compositions having a weight ratio of exosome:TSP of 1:2500 to 1:10000.

In some embodiments, the exosome compositions comprise an exosome:TSP weight ratio of about 1:2500, 1:3000, 1:4000, 1:5000, 1:5500, 1:6000, 1:6500, 1:7000, 1:8000, 1:9000, or 1:10000.

In some embodiments, the exosome compositions comprise an exosome:TSP weight ratio of at least 1:2500, 1:3000, 1:4000, 1:5000, 1:5500, 1:6000, 1:6500, 1:7000, 1:8000, 1:9000, or 1:10000.

In some embodiments, the exosome compositions comprise an exosome:TSP weight ratio of at least about 1:2500, 1:3000, 1:4000, 1:5000, 1:5500, 1:6000, 1:6500, 1:7000, 1:8000, 1:9000, or 1:10000.

### TSP Sequences

The TSPs of the present technology may comprise or consist of non-naturally occurring amino acid sequences. The amino acid sequence may be about 4 to about 15 amino acids in length.

In some embodiments, TSPs comprise or consist of an amino acid sequence at least 4 to about 15 amino acids in length.

In some embodiments, the TSPs comprise or consist of an amino acid sequence at least 4 to at least 15 amino acids in length.

TSPs may comprise or consist of an antibody or an antigen-binding fragment thereof or an aptamer (e.g., an RNA-comprising aptamer). Nonlimiting examples of antigen-binding fragments include a variable region of an antibody heavy chain and/or light chain.

### TSP Targets

The TSPs of the present technology may at least partially bind a cell surface protein, such as a transmembrane protein, or a peptide thereof of a cancer cell or otherwise within a tumor microenvironment. In some embodiments, the transmembrane protein is an integrin (i.e., “an integrin receptor”), or a subunit thereof. The integrin may be selected from the group consisting of an arginine-glycine-aspartic acid (RGD)-binding integrin, a collagen-binding integrin, a laminin-binding integrin, and a leukocyte-specific integrin. In some embodiments, the integrin comprises or consists of αvβ1, αvβ3, αvβ5, αvβ6, αvβ8, α8β1, αIIbβ3, α1β1, α2β1, α3β1, α10β1, α11β1, α6β1, α7β1, α6β4, αLβ2, αMβ2, αXβ2, αDβ2, α4β1, α4β7, α9β1, or αEβ7.

In some embodiments, the TSP specifically binds at least a portion of an integrin subunit selected from the group consisting of alpha I (National Center for Biotechnology Information (NCBI) Accession(s): KAI4021230.1, KAI4021231.1), alpha 2 (NCBI Accession(s): KAI4021236.1, KAI4021234.1, KAI4021233.1, KAI4021235.1, KAI4021232.1, KAI4021237.1, EAW54873.1), alpha Ilb (NCBI Accession(s): P08514.3, 3FCS_C, 3FCS_A, 3FCU_E), alpha 3 (NCBI Accession(s): KAI4050412.1, KAI4050411.1, KAI4050410.1), alpha 4 (NCBI Accession(s): KAI4037171.1, KAI2526017.1, KAI4037170.1, AAB59613.1, EAX10987.1, EAX10986.1), alpha V (NCBI Accession(s): KAI4037224.1, KAI4037223.1, KAI4037222.1, P06756.2, P08648.2), alpha 8 (NCBI Accession(s): KAI4075339.1), alpha 6 (NCBI Accession(s): KAI4036941.1, KAI4036940.1, KAI4036944.1, KAI4036943.1, KAI4036942.1, KAI2525782.1, CAA37655.1), alpha 7 (NCBI Accession(s): KAI4066426.1, KAI4066425.1, KAI4066424.1, KAI4066423.1, KAI4066422.1, KAI4066430.1, KAI4066428.1), alpha 9 (NCBI Accession(s): KAI4028899.1, KAI4028898.1), alpha 10 (NCBI Accession(s): KAI4082275.1, KAI402274.1, EAW71424.1), alpha 11 (NCBI Accession(s): KAI4058464.1, KAI4058463.1), alpha E (NCBI Accession(s): KAI4047160.1, CAA37655.1), alpha D (NCBI Accession(s): KAI4054699.1, BAD92182.1, EAW52139.1), alpha L (NCBI Accession(s): KAI4054561.1, KAI4054560.1, KAI4054566.1, KAI4054564.1, KAI4054563.1, KAI4054562.1, KAI4054559.1), M (NCBI Accession(s): KAI4054694.1, KAI4054693.1), alpha X (NCBI Accession(s): KAI4054698.1, KAI4054697.1, KAI4054696.1, EAW52141.1, EAW52140.1, EAW52142.1), beta I (NCBI Accession(s): KAI4075647.1, KAI4075646.1), beta 2 (NCBI Accession(s): KAI4004316.1, KAI4004314.1, KAI4004312.1, KAI4004323.1, KAI4004321.1, KAI4004315.1, KAI2596459.1, KAI2596457.1), beta 3 (NCBI Accession(s): KAI4050162.1, KAI4050163.1, P05106.2), beta 4 (NCBI Accession(s): KAI4051610.1, KAI4051609.1, KAI4051608.1), beta 5 (NCBI Accession: KAI4031295.1), beta 6 (NCBI Accession(s): CAA01832.1, KAI4036632.1, KAI4036631.1, KAI2525470, KAI4036630.1), beta 7 (NCBI Accession(s): KAI4066171.1, KAI4066172.1, KAI4066173.1), beta 81 (NCBI Accession(s): KAI4013063.1, KAI4013062.1).

The TSPs of the present technology may comprise or consist of an RGD motif that binds to at least a portion of the integrin. In some embodiments, the TSP comprises or consists of an internalizing RGD (iRGD) peptide.

In some embodiments, the TSP comprising an RGD motif comprises an amino acid sequence of CRGDKGPDC (SEQ ID NO: 1).

In some embodiments, the TSP comprising an RGD motif comprises an amino acid sequence of RGDKGPDC (SEQ ID NO: 2).

In some embodiments, a TSP of the present technology comprises or consists of an amino acid sequence that is about 100%, 99%, 98%, 95%, 90%, 85%, 80%, 75%, 70%, 65%, 60%, or 50% identical to the amino acid sequence of SEQ ID NO: 1 or SEQ ID NO: 2.

In some embodiments, a TSP of the present technology comprises or consists of an amino acid sequence that is at least 100%, 99%, 98%, 95%, 90%, 85%, 80%, 75%, 70%, 65%, 60%, or 50% identical to the amino acid sequence of SEQ ID NO: 1 or SEQ ID NO: 2.

In some embodiments, a TSP of the present technology comprises or consists of an amino acid sequence that is at least about 100%, 99%, 98%, 95%, 90%, 85%, 80%, 75%, 70%, 65%, 60%, or 50% identical to the amino acid sequence of SEQ ID NO: 1 or SEQ ID NO: 2.

### Additional TSP Features

The TSPs of the present technology may be utilized to tag an exosome. For example, the TSPs of the present technology may be conjugated to a lipoprotein for tagging (or otherwise incorporating) an exosome with additional cancer cell specificity. Tagging of the exosomes may enable targeting of additional cancer cells or peptides thereof for exosome delivery, such as dual targeting of αvβ3 and αvβ5 integrins.

### TSP Conjugates

In some embodiments, the TSPs of the present technology comprise conjugate, such as a lipid-polymer conjugate, which at least partially conjugates the TSP to the exosome. The lipid-polymer conjugate may be used to functionalize exosomes and/or to enable click chemistry attachment of the TSPs within the exosome compositions of the present technology. The lipid-polymer conjugate may comprise or consist of one or more of a phospholipid, a polymer, and a functional group.

The phospholipid may be used to insert the TSP into lipid bilayers (e.g., an exosome lipid bilayer). In some embodiments, the phospholipid is 1,2-distearoyl-sn-glycero-3-phosphoethanolamine (DSPE).

The polymer may be a hydrophilic polymer. The polymer may be used to (i) project the functional group away from a membrane surface, such as that of the exosomes, (ii) improve colloidal stability and/or reduce nonspecific interactions of the conjugate, relative to a control, and/or (iii) provide flexibility or steric accessibility for binding of the TSP and/or conjugate, relative to a control. In some embodiments, the polymer is poly(ethylene glycol) (PEG5000).

The functional group may be a terminal functional group. The functional group may be utilized for click reactions (e.g., biorthogonal click reactions), such as strain-promoted azide-alkyne cycloaddition (SPAAC) with DBCO/BCN-alkyne ligands (copper-free), or copper (I)-catalyzed azide-alkyne cycloaddition (CuAAC) with terminal alkynes (copper-catalyzed). In some embodiments, the functional group is an azide functional group

In some embodiments, the conjugate comprises or consists of DSPE-PEG5000-N3.

### Anticancer Agents

The TSP-Exos of the present technology may at least partially encapsulate one or more anticancer agents within the exosome for targeted delivery of the anticancer agents to the cancer cells and/or the tumor microenvironment of the present technology. Nonlimiting examples of suitable anticancer agents include alkylating agents, platinum agents, antimetabolites, topoisomerase inhibitors (e.g., topoisomerase I or topoisomerase II inhibitors), microtubule-targeting agents, antitumor antibiotics, biologics, and other cytotoxic agents.

The alkylating agent may be selected from the group consisting of cyclophosphamide, ifosfamide, melphalan, and bendamustine.

The platinum agent may be selected from the group consisting of cisplatin, carboplatin, and oxaliplatin.

The antimetabolite may be selected from the group consisting of 5-fluorouracil (5-FU), capecitabine, methotrexate, cytarabine, gemcitabine, and pemetrexed.

The topoisomerase inhibitor may be selected from the group consisting of doxorubicin (DOX), epirubicin, irinotecan, topotecan, etoposide, and teniposide.

The microtubule-targeting agent may be selected from the group consisting of paclitaxel, docetaxel, vincristine, vinblastine, vinorelbine, and cabazitaxel. In some embodiments, the microtubule-targeting agent includes taxanes, vinca alkaloids, and other microtubule inhibitors.

The antitumor antibiotic may be selected from dactinomycin and mitomycin.

The biologic may be selected from the group consisting of dactinomycin and mitomycin. bortezomib, carfilzomib, imatinib, bosutinib, erlotinib, cetuximab, panitumumab, bevacizumab, sorafenib, sunitinib, pazopanib, and ziv-aflibercept.

The cytotoxic agent may be bleomycin, dacarbazine, or temozolomide. In some embodiments, the cytotoxic agent is an anthracycline/anthracenedione, which may be selected from DOX, epirubicin, daunorubicin, idarubicin, and mitoxantrone.

In some embodiments, the TSP-Exos comprise DOX at least partially encapsulated by the exosome.

In some embodiments, the at least partial encapsulation includes full encapsulation of the one or more anticancer agents.

In some embodiments, the one or more anticancer agents comprise or consist of two or more anticancer agents.

The one or more anticancer agents may be present in the exosomes of the present technology in an amount of about 10 ng/ml to about 30 mg/mL.

The one or more anticancer agents may be present in the exosomes of the present technology in an amount of about 10 ng/ml, 20 ng/ml, 50 ng/mL, 100 ng/ml, 200 ng/ml, 500 ng/ml, 1 μg/mL, 2 μg/mL, 5 μg/mL, 10 μg/mL, 20 μg/mL, 50 μg/mL, 100 μg/mL, 200 μg/mL, 500 μg/mL, 1 mg/mL, 1.5 mg/mL, 2 mg/mL, 3 mg/mL, 5 mg/mL, 10 mg/mL, 15 mg/mL, or 20 mg/mL.

The one or more anticancer agents may be present in the exosomes of the present technology in an amount of at least 10 ng/ml, 20 ng/ml, 50 ng/ml, 100 ng/ml, 200 ng/ml, 500 ng/ml, 1 μg/mL, 2 μg/mL, 5 μg/mL, 10 μg/mL, 20 μg/mL, 50 μg/mL, 100 μg/mL, 200 μg/mL, 500 μg/mL, 1 mg/mL, 1.5 mg/mL, 2 mg/mL, 3 mg/mL, 5 mg/mL, 10 mg/mL, 15 mg/mL, or 20 mg/mL.

The one or more anticancer agents may be present in the exosomes of the present technology in an amount of at least about 10 ng/ml, 20 ng/ml, 50 ng/ml, 100 ng/ml, 200 ng/ml, 500 ng/ml, 1 μg/mL, 2 μg/mL, 5 μg/mL, 10 μg/mL, 20 μg/mL, 50 μg/mL, 100 μg/mL, 200 μg/mL, 500 μg/mL, 1 mg/mL, 1.5 mg/mL, 2 mg/mL, 3 mg/mL, 5 mg/mL, 10 mg/mL, 15 mg/mL, or 20 mg/mL.

In some embodiments, the one or more anticancer agents are at least partially encapsulated by the exosome in an amount of about 10 μg/mg, 15 μg/mg, 20 μg/mg, 25 μg/mg, 30 μg/mg, 35 μg/mg, or 40 μg/mg, relative to the amount of exosomes.

In some embodiments, the one or more anticancer agents are at least partially encapsulated by the exosome in an amount of at least 10 μg/mg, 15 μg/mg, 20 μg/mg, 25 μg/mg, 30 μg/mg, 35 μg/mg, or 40 μg/mg, relative to the amount of exosomes.

In some embodiments, the one or more anticancer agents are at least partially encapsulated by the exosome in an amount of at least about 10 μg/mg, 15 μg/mg, 20 μg/mg, 25 μg/mg, 30 μg/mg, 35 μg/mg, or 40 μg/mg, relative to the amount of exosomes.

The anticancer agents may be loaded into the exosome compositions with a weight ratio of TSP and exosomes:anticancer agent of 10:1 to 20:1.

In some embodiments the anticancer agents may be loaded into the exosome compositions with a weight ratio of TSP and exosomes:anticancer agent of about 10:1, 11:1, 12:1, 13:1, 14:1, 15:1, 16:1, 17:1, 18:1, 19:1, or 20:1.

In some embodiments the anticancer agents may be loaded into the exosome compositions with a weight ratio of TSP and exosomes:anticancer agent of at least 10:1, 11:1, 12:1, 13:1, 14:1, 15:1, 16:1, 17:1, 18:1, 19:1, or 20:1.

In some embodiments the anticancer agents may be loaded into the exosome compositions with a weight ratio of TSP and exosomes:anticancer agent of at least about 10:1, 11:1, 12:1, 13:1, 14:1, 15:1, 16:1, 17:1, 18:1, 19:1, or 20:1.

In some embodiments, DOX is loaded into the exosome compositions with a weight ratio of TSP and exosomes:DOX of about 14:1.

In some embodiments, the one or more anticancer agents are at least partially encapsulated by the exosome using ultrasonication or electroporation.

### Methods

### Cancer Associated Methods

The exosome compositions of the present technology may be useful in targeting cancer in subjects in need thereof. Nonlimiting examples of cancer include breast cancer, lung cancer, colorectal cancer, prostate cancer, pancreatic cancer, ovarian cancer, cervical cancer, endometrial cancer, liver cancer, gastric cancer, esophageal cancer, kidney cancer, bladder cancer, melanoma, thyroid cancer, head and neck cancer, brain cancer, testicular cancer, sarcoma, bone cancer, lymphoma, multiple myeloma, and leukemia. In some embodiments, the breast cancer is TNBC. The exosome compositions may be useful in treating cancer in subjects in need thereof. In some embodiments, treating cancer includes reducing one or more of a tumor size, a tumor volume, and a cancer metastasis level.

The present technology comprises methods of enhancing one or more anticancer agent effects against one or more cancer cells in a subject in need thereof, relative to a control, comprising administering to the subject, an exosome composition of the present technology. Enhancing one or more anticancer agent effects includes, but is not limited to, reducing or preventing an increase in a tumor size, reducing or preventing an increase in a tumor volume, and/or reducing or preventing a cancer metastasis level in a subject in need thereof, relative to a control. The control may comprise the anticancer agent administered alone or within a composition lacking one or more features of the exosome compositions of the present technology.

In some embodiments, the methods of the present technology comprise reducing or preventing an anticancer agent toxicity level in a subject in need thereof, relative to a control, comprising administering to the subject, an exosome composition of the present technology. Nonlimiting examples of anticancer agent toxicity include anticancer agent-induced cardiotoxicity and cell death in non-cancer cell populations. In some embodiments, the cardiotoxicity is cardiomyopathy, such as DOX-induced cardiomyopathy (DIC). DIC and other cardiotoxicities may comprise and/or be measured by one or more of an increase in cardiac fibrosis, cytoplasmic vacuolization, or cardiac apoptosis, relative to a control. In some embodiments, the methods reduce or prevent an anticancer agent toxicity level without altering efficacy of the anticancer agent.

The exosome compositions of the present technology may be useful in one or more of (a)-(h):
(a) reducing or preventing an increase in a tumor size, relative to a control;
(b) reducing or preventing an increase in a tumor volume in the subject, relative to a control;
(c) increasing a level of cancer cell death in the subject, relative to a control;
(d) reduction in a cell death level of a non-cancer cell population in the subject, relative to a control;
(e) reducing or preventing a cancer metastasis level in the subject, relative to the control;
(f) reducing an anticancer agent toxicity level in the subject, relative to a control;
(g) reducing or preventing an anticancer agent induced cardiotoxicity in the subject, relative to a control; and
(h) increasing an anticancer agent efficacy level in the subject, relative to a control.

Any one of (a)-(h) may occur about 30 minutes, 1 hour, 2 hours, 3 hours, 4 hours, 5 hours, 6 hours, 8 hours, 12 hours, 1 day, 2 days, 3 days, 4 days, 1 week, 2 weeks, 3 weeks, 4 weeks, 1 month, or 2 months after administration of a first dose of the exosome compositions of the present technology.

Any one of (a)-(h) may occur at least 30 minutes, 1 hour, 2 hours, 3 hours, 4 hours, 5 hours, 6 hours, 8 hours, 12 hours, 1 day, 2 days, 3 days, 4 days, 1 week, 2 weeks, 3 weeks, 4 weeks, 1 month, or 2 months after administration of a first dose of the exosome compositions of the present technology.

Any one of (a)-(h) may occur at least about 30 minutes, 1 hour, 2 hours, 3 hours, 4 hours, 5 hours, 6 hours, 8 hours, 12 hours, 1 day, 2 days, 3 days, 4 days, 1 week, 2 weeks, 3 weeks, 4 weeks, 1 month, or 2 months after administration of a first dose of the exosome compositions of the present technology.

In some embodiments, the methods of the present technology comprise reducing a tumor size by about 5%, 10%, 15%, 20%, 25%, 30%, 40%, 50%, 60%, 70%, 80%, 90%, 95%, 98%, 99%, or 100%, relative to a control.

In some embodiments, the methods of the present technology comprise reducing a tumor size by at least 5%, 10%, 15%, 20%, 25%, 30%, 40%, 50%, 60%, 70%, 80%, 90%, 95%, 98%, 99%, or 100%, relative to a control.

In some embodiments, the methods of the present technology comprise reducing a tumor size by at least about 5%, 10%, 15%, 20%, 25%, 30%, 40%, 50%, 60%, 70%, 80%, 90%, 95%, 98%, 99%, or 100%, relative to a control.

In some embodiments, the methods of the present technology comprise reducing a tumor volume by about 5%, 10%, 15%, 20%, 25%, 30%, 40%, 50%, 60%, 70%, 80%, 90%, 95%, 98%, 99%, or 100%, relative to a control.

In some embodiments, the methods of the present technology comprise reducing a tumor volume by at least 5%, 10%, 15%, 20%, 25%, 30%, 40%, 50%, 60%, 70%, 80%, 90%, 95%, 98%, 99%, or 100%, relative to a control.

In some embodiments, the methods of the present technology comprise reducing a tumor volume by at least about 5%, 10%, 15%, 20%, 25%, 30%, 40%, 50%, 60%, 70%, 80%, 90%, 95%, 98%, 99%, or 100%, relative to a control.

In some embodiments, the methods of the present technology comprise increasing a level of cancer cell death by about 5%, 10%, 15%, 20%, 25%, 30%, 40%, 50%, 60%, 70%, 80%, 90%, 100%, 150%, 200%, 300%, 400%, or 500%, relative to a control.

In some embodiments, the methods of the present technology comprise increasing a level of cancer cell death by at least 5%, 10%, 15%, 20%, 25%, 30%, 40%, 50%, 60%, 70%, 80%, 90%, 100%, 150%, 200%, 300%, 400%, or 500%, relative to a control.

In some embodiments, the methods of the present technology comprise increasing a level of cancer cell death by at least about 5%, 10%, 15%, 20%, 25%, 30%, 40%, 50%, 60%, 70%, 80%, 90%, 100%, 150%, 200%, 300%, 400%, or 500%, relative to a control.

In some embodiments, the methods of the present technology comprise reducing a cell death level of a non-cancer cell population by about 5%, 10%, 15%, 20%, 25%, 30%, 40%, 50%, 60%, 70%, 80%, 90%, 95%, 98%, 99%, or 100%, relative to a control.

In some embodiments, the methods of the present technology comprise reducing a cell death level of a non-cancer cell population by at least 5%, 10%, 15%, 20%, 25%, 30%, 40%, 50%, 60%, 70%, 80%, 90%, 95%, 98%, 99%, or 100%, relative to a control.

In some embodiments, the methods of the present technology comprise reducing a cell death level of a non-cancer cell population by at least about 5%, 10%, 15%, 20%, 25%, 30%, 40%, 50%, 60%, 70%, 80%, 90%, 95%, 98%, 99%, or 100%, relative to a control.

In some embodiments, the methods of the present technology comprise reducing a cancer metastasis level by about 5%, 10%, 15%, 20%, 25%, 30%, 40%, 50%, 60%, 70%, 80%, 90%, 95%, 98%, 99%, or 100%, relative to a control.

In some embodiments, the methods of the present technology comprise reducing a cancer metastasis level by at least 5%, 10%, 15%, 20%, 25%, 30%, 40%, 50%, 60%, 70%, 80%, 90%, 95%, 98%, 99%, or 100%, relative to a control.

In some embodiments, the methods of the present technology comprise reducing a cancer metastasis level by at least about 5%, 10%, 15%, 20%, 25%, 30%, 40%, 50%, 60%, 70%, 80%, 90%, 95%, 98%, 99%, or 100%, relative to a control.

In some embodiments, the methods of the present technology comprise reducing an anticancer agent toxicity level in the subject, by about 5%, 10%, 15%, 20%, 25%, 30%, 40%, 50%, 60%, 70%, 80%, 90%, 95%, 98%, 99%, or 100%, relative to a control.

In some embodiments, the methods of the present technology comprise reducing an anticancer agent toxicity level in the subject, by at least 5%, 10%, 15%, 20%, 25%, 30%, 40%, 50%, 60%, 70%, 80%, 90%, 95%, 98%, 99%, or 100%, relative to a control.

In some embodiments, the methods of the present technology comprise reducing an anticancer agent toxicity level in the subject, by at least about 5%, 10%, 15%, 20%, 25%, 30%, 40%, 50%, 60%, 70%, 80%, 90%, 95%, 98%, 99%, or 100%, relative to a control.

In some embodiments, the methods of the present technology comprise reducing an anticancer agent induced cardiotoxicity in the subject, by about 5%, 10%, 15%, 20%, 25%, 30%, 40%, 50%, 60%, 70%, 80%, 90%, 95%, 98%, 99%, or 100%, relative to a control.

In some embodiments, the methods of the present technology comprise reducing an anticancer agent induced cardiotoxicity in the subject, by at least 5%, 10%, 15%, 20%, 25%, 30%, 40%, 50%, 60%, 70%, 80%, 90%, 95%, 98%, 99%, or 100%, relative to a control.

In some embodiments, the methods of the present technology comprise reducing an anticancer agent induced cardiotoxicity in the subject, by at least about 5%, 10%, 15%, 20%, 25%, 30%, 40%, 50%, 60%, 70%, 80%, 90%, 95%, 98%, 99%, or 100%, relative to a control.

In some embodiments, the methods of the present technology comprise increasing an anticancer agent efficacy level by about 5%, 10%, 15%, 20%, 25%, 30%, 40%, 50%, 60%, 70%, 80%, 90%, 100%, 150%, 200%, 300%, 400%, or 500%, relative to a control.

In some embodiments, the methods of the present technology comprise increasing an anticancer agent efficacy level by at least 5%, 10%, 15%, 20%, 25%, 30%, 40%, 50%, 60%, 70%, 80%, 90%, 100%, 150%, 200%, 300%, 400%, or 500%, relative to a control.

In some embodiments, the methods of the present technology comprise increasing an anticancer agent efficacy level by at least about 5%, 10%, 15%, 20%, 25%, 30%, 40%, 50%, 60%, 70%, 80%, 90%, 100%, 150%, 200%, 300%, 400%, or 500%, relative to a control.

The level of cancer cell death or the cell death level of the non-cancer cell population of the methods of the present technology may include apoptosis and/or necrosis. Any of cell death of the present technology may be assessed by any means known in the art, including but not limited to cell staining (e.g., TUNEL staining), cell viability assays, and cell death expression markers (e.g., BAX/Bcl2 and/or Caspase-3 gene expression or protein levels).

In some embodiments, the non-cancer cell population comprises one or more cardiac cells. Nonlimiting examples of cardiac cells include cardiomyocytes.

The cardiotoxicity of the methods of the present technology may be assessed by any means known in the art, including assessment of one or more of ejection fraction, fractional shortening, ventricular mass, and markers of cardiac function (e.g., assessment of natriuretic peptide A (ANP; NCBI Gene ID: 4878), natriuretic peptide B (BNP; NCBI Gene ID: 4879), and/or myosin heavy chain 7 (MYH7; NCBI Gene ID: 4625) gene expression levels).

### Subjects

The subject of the present technology may have or be at risk of developing a cancer. In some embodiments, the subject has TNBC. In some embodiments the subject has or is at risk of developing primary or metastatic breast cancer.

In some embodiments, the subject is human or murine.

The subject may be a male or female subject. In some embodiments, the subject is an adult subject or an adolescent subject.

In some embodiments, the subject has received or is receiving an anticancer agent. In some embodiments, the subject has not received an anticancer agent. In some embodiments, the subject has received or is receiving a cancer treatment selected from the group consisting of immunotherapy, radiation therapy, and surgical therapy.

### Formulations and Administration

### Pharmaceutical Compositions

The exosome compositions of the present technology (e.g., TSP-Exos, including those comprising one or more anticancer agents) may be present in a pharmaceutical composition.

The exosome compositions may be formulated with one or more pharmaceutically acceptable carriers and/or excipients. The carriers and/or excipients of the present technology facilitate delivery of the exosome composition to a subject. Other pharmaceutically acceptable carriers and/or excipients may be included in the pharmaceutical composition to enhance dispersion, solubility, and/or stability of the exosome composition or any of its components, and/or to reduce adverse injection site reactions.

In some embodiments, the pharmaceutical composition comprises 0.1 to 99.9999 wt. %, 1 to 99.999 wt. %, 5 to 99.99 wt. %, 10 to 99.9 wt. %, 15 to 99 wt. %, 20 to 90 wt. %, 30 to 85 wt. %, 40 to 80 wt. %, 50 to 75 wt. %, or 60 to 70 wt. % of the pharmaceutically acceptable carrier and/or excipient relative to a total weight of the pharmaceutical composition.

In some embodiments, the pharmaceutically acceptable carrier and/or excipient is selected from the group consisting of water, a buffer, an inorganic salt, a fatty acid, a vegetable oil, a synthetic fatty ester, a surfactant, and a polymer. The water may act as a diluent and include, without limitation, water for injection (WFI), sterile water, bacteriostatic water for injection (BWFI), distilled water, bidistilled water, deionized water, deionized distilled water, and reverse osmosis water. In some embodiments, the water is for injection.

In some embodiments, the pharmaceutically acceptable carrier and/or excipient is water. In some embodiments, the pharmaceutically acceptable carrier and/or is a buffer.

The pharmaceutically acceptable excipients may be modified for each dosage form. For example, parenteral administration, the pharmaceutical composition may comprise lyoprotectants (e.g., human albumin, lactose monohydrate, maltose/trehalose/sucrose, mannitol, dextran, inulin, fructose), micro-encapsulating agents (e.g., aliphatic polyester such as polyglycolide, polylactide, and their copolymers, phospholipids/lecithin, phosphatidic acids, phosphoglycerol, phosphoserine, phosphorethanolamine, phosphocholine, PEGylated phospholipids, hydroxypropylcyclodextrin, Betadex sulfobutyl ether sodium), solubilizers and emulsifiers (e.g., N-methyl 2-pyrrolidone, PEG, polysorbates, polyoxyl 35 castor oil, polyoxyl-15-hydroxystearate, polyvinyl pyrrolidone, propylene glycol, sodium cholesteryl sulfate, sorbitan esters, poloxamer, 2-pyrrolidone, diacylglycerols, monglycerol), tonicity agents (e.g., dextrose, glycerin, mannitol, NaCl, KCl, sorbitol/sorbitol solution), solvents and cosolvents (water miscible such as propylene glycol, PEG low molecular weight, glycerin, ethanol, 2-pyrrolidone, and N-methyl-2-pyrrolidone and water immiscible such as ethyl oleate, benzyl benzoate, vegetable oil, soybean oil, sesame oil, peanut oil, castor oil, almond oil, and cottonseed oil), viscosity-building agents (e.g., sodium carboxymethylcellulose (Na CMC), methylcellulose, gelatin, polyvinyl pyrrolidone), antioxidants (e.g., ascorbic acid, acetylcysteine, sodium ascorbate, sodium metabisulfite, sodium bisulfite, and tocopherol), chelating agents (e.g., EDTA), preservatives (e.g., methyl paraben, ethyl paraben, propyl paraben, butyl paraben, benzoic acid, sodium benzoate, EDTA, cetrimide, benzyl alcohol, benzalkonium chloride, thimerosal, and phenylmercuric salts), buffering agents (e.g., acetate, citrate, tartrate, phosphate, triethanolamine (TRIS) buffer). Examples of pharmaceutical excipients for other dosage forms may be found in Remington: The Science & Practice of Pharmacy 23^{rd} Edition, Elsevier, the disclosure of which is incorporated herein by reference in its entirety.

### Administration

The exosome compositions of the present technology may be formulated for administration to a subject in need thereof. In some embodiments, the exosome compositions are formulated for administration through any means known in the art, including orally, rectally, vaginally, ocularly, intranasally, topically, parenterally, or by injection. If administered by injection, the injection may be intravenous (IV), subcutaneous (SC), intramuscular (IM), intraperitoneal (IP), intracerebroventricular (ICV), or other means known in the art.

The pharmaceutical compositions may be formulated by any means known in the art, including but not limited to formulation as tablets, capsules, caplets, suspensions, powders, lyophilized preparations, suppositories, pessaries, ocular drops, skin patches, orally soluble formulations, enteric formulations, solutions sprays, aerosols and the like, and may be mixed and formulated with buffers, binders, excipients, stabilizers, lubricants, oils, adjuvants, anti-oxidants and other agents known in the art. In general, any route of administration by which the peptides are introduced across an epidermal layer of cells may be employed. Administration includes topical delivery. Administration includes delivery across the blood brain barrier. Administration includes delivery through mucous membranes, buccal administration, ophthalmic administration, oral administration, dermal administration, inhalation administration, nasal administration, urethral administration, vaginal administration, rectal administration, and the like.

In some embodiments, the exosome compositions of the present technology are formulated for administration from a surgical device. In some embodiments, the device is a surgically implanted device. The device may be utilized for controlled release of the exosome compositions of the present technology to tumor tissues or organs. In some embodiments, the device is an implantable drug delivery system (“IDDS”, e.g., biodegradable IDDS, non-biodegradable IDDS, and refillable IDDs). The IDDs may be implanted to a desired area, such as brain or spine implantation.

Compositions for each administration route may be formulated into a proper dosage form and comprise any necessary excipients. For example, a composition of the present technology may be dissolved or suspended in saline or other buffers to be manufactured as a solution for nebulizer mist, nasal spray, or intravenous injection, and each of solutions for different route administration may have similar or different excipients. Compositions of the present technology may also be formulated into powder or granules to be dissolved later with a proper buffer solution.

In some embodiments, the exosome compositions of the present technology are formulated for delivery to a subject in need thereof before, during, or after the subject is administered an anticancer agent of the present technology or other cancer treatments (e.g., radiation therapy, immunotherapy, surgical intervention). In some embodiments, the exosome compositions are formulated for co-administration with an anti-cancer agent or other cancer treatments. The anticancer agent may be a chemotherapy.

### Dosing

The exosome compositions of the present technology may be formulated for administration to a subject in need thereof for daily, weekly, twice weekly, or three times weekly administration. The exosome compositions may be administered for one day, two days, three days, 4 days, 1 week, 10 days, 2 weeks, 3 weeks, 4 weeks, 1 month, two months, three months, four months, five months, six months, 1 year, or longer. Doses may be determined empirically, depending on the severity or the type of cancer. Administration and dosing may be determined as needed based on tolerability to the exosome compositions of the present technology.

In some embodiments, the exosome compositions are administered to the subject in an amount of about 1.5 mg/kg/day to about 45 mg/kg/day.

In some embodiments, the exosome compositions are administered to the subject in an amount of about 1.5 mg/kg/day, about 2.5 mg/kg/day, about 5 mg/kg/day, about 7.5 mg/kg/day, about 10 mg/kg/day, about 12.5 mg/kg/day, about 15 mg/kg/day, about 20 mg/kg/day, about 25 mg/kg/day, about 30 mg/kg/day, about 35 mg/kg/day, about 40 mg/kg/day, or about 45 mg/kg/day.

In some embodiments, the exosome compositions are administered to the subject in an amount of at least 1.5 mg/kg/day, at least 2.5 mg/kg/day, at least 5 mg/kg/day, at least 7.5 mg/kg/day, at least 10 mg/kg/day, at least 12.5 mg/kg/day, at least 15 mg/kg/day, at least 20 mg/kg/day, at least 25 mg/kg/day, at least 30 mg/kg/day, at least 35 mg/kg/day, at least 40 mg/kg/day, or at least 45 mg/kg/day.

In some embodiments, the exosome compositions are administered to the subject in an amount of at least about 1.5 mg/kg/day, at least about 2.5 mg/kg/day, at least about 5 mg/kg/day, at least about 7.5 mg/kg/day, at least about 10 mg/kg/day, at least about 12.5 mg/kg/day, at least about 15 mg/kg/day, at least about 20 mg/kg/day, at least about 25 mg/kg/day, at least about 30 mg/kg/day, at least about 35 mg/kg/day, at least about 40 mg/kg/day, or at least about 45 mg/kg/day.

In some embodiments, the exosome compositions are administered to the subject to provide an amount of the one or more anticancer agents that is about 0.01 mg/kg/day to about 10.0 mg/kg/day.

In some embodiments, the exosome compositions are administered to the subject to provide an amount of the one or more anticancer agents that is about 0.01 mg/kg/day, about 0.05 mg/kg/day, about 0.1 mg/kg/day, about 0.25 mg/kg/day, about 0.5 mg/kg/day, about 1.0 mg/kg/day, about 2.5 mg/kg/day, about 5.0 mg/kg/day, about 7.5 mg/kg/day, or about 10.0 mg/kg/day.

In some embodiments, the exosome compositions are administered to the subject to provide an amount of the one or more anticancer agents that is at least 0.01 mg/kg/day, at least 0.05 mg/kg/day, at least 0.1 mg/kg/day, at least 0.25 mg/kg/day, at least 0.5 mg/kg/day, at least 1.0 mg/kg/day, at least 2.5 mg/kg/day, at least 5.0 mg/kg/day, at least 7.5 mg/kg/day, or at least 10.0 mg/kg/day.

In some embodiments, the exosome compositions are administered to the subject to provide an amount of the one or more anticancer agents that is at least about 0.01 mg/kg/day, at least about 0.05 mg/kg/day, at least about 0.1 mg/kg/day, at least about 0.25 mg/kg/day, at least about 0.5 mg/kg/day, at least about 1.0 mg/kg/day, at least about 2.5 mg/kg/day, at least about 5.0 mg/kg/day, at least about 7.5 mg/kg/day, or at least about 10.0 mg/kg/day.

### EXAMPLES

The following examples are intended to illustrate various embodiments of the present technology. As such, the specific embodiments discussed are not to be construed as limitations on the scope of the present technology. It will be apparent to one skilled in the art that various equivalents, changes, and modifications may be made without departing from the scope of present technology, and it is understood that such equivalent embodiments, are to be included herein. Further, all references cited herein are hereby incorporated by reference in their entirety, as if fully set forth herein.

### Example 1: Mesenchymal Stem Cell-Exosome (MSc-Exos) Isolation and Characterization

To synthesize and assess the exosome compositions of the present technology, MSc-Exos were generated. Exosomes were isolated from murine MScs using ultrafiltration methods described in Shu S et al., A Rapid Exosome Isolation Using Ultrafiltration and Size Exclusion Chromatography (REIUS) Method for Exosome Isolation from Melanoma Cell Lines. Methods Mol Biol 2021, 2265:289-304, the disclosure of which is incorporated herein by reference in its entirety.

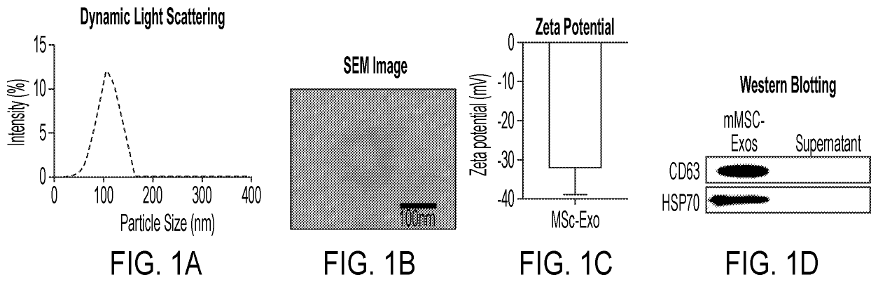

Isolated exosomes were characterized using four different methods: (1) particle size and distribution (using dynamic light scattering, DLS), (2) morphology (scanning electron microscopy (SEM)), (3) zeta potential (Zetasizer nano series instrument, and 4) Western blotting. Particle size and distribution of isolated exosomes were within the range of 50-150 nM (FIG. 1A). Furthermore, the isolated exosomes' morphology (FIG. 1B) using SEM and negative zeta potential (FIG. 1C) corroborated with exosomes as previously described (see Sokolova V et al., Characterisation of exosomes derived from human cells by nanoparticle tracking analysis and scanning electron microscopy. Colloids Surf B Biointerfaces 2011, 87:146-50 and Li S et al., Mesenchymal stem cell-derived extracellular vesicles prevent the development of osteoarthritis via the circHIPK3/miR-124-3p/MYH9 axis. J Nanobiotechnology 2021, 19:194, the disclosures of each are incorporated herein by reference in their entireties). Further analysis confirmed presence of isolated exosomal markers, CD63 and HSP70 (FIG. 1D).

### Example 2: Generation of Tumor-Specific Peptide (TSP)-iRGD Tagged and Fluorescence Labelled MSc-Exosomes

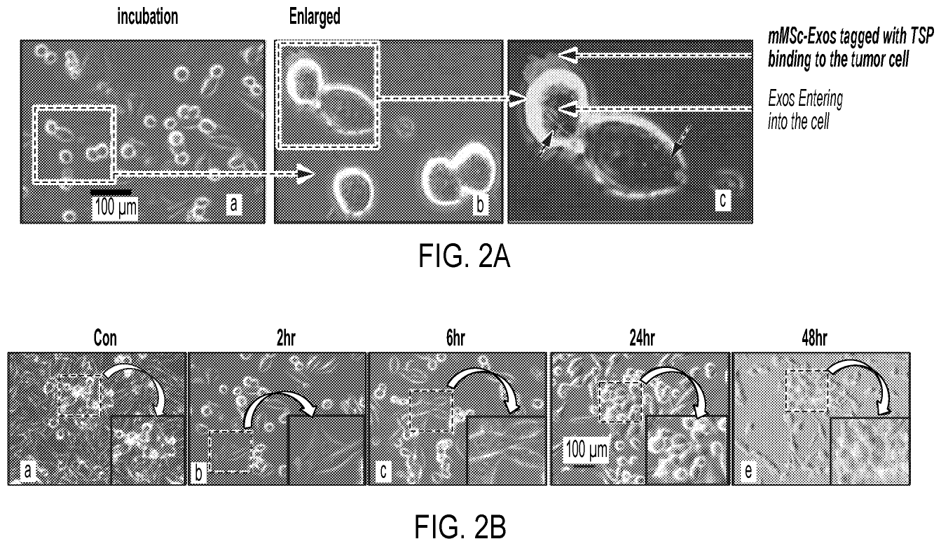

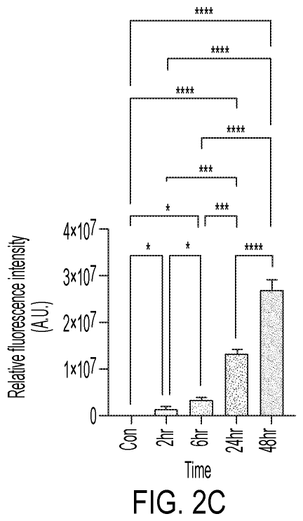

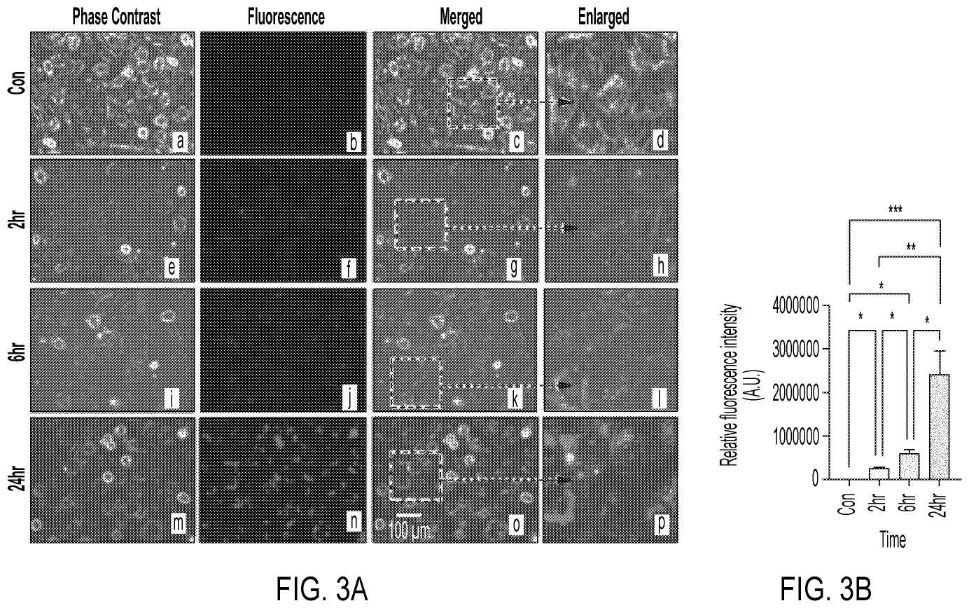

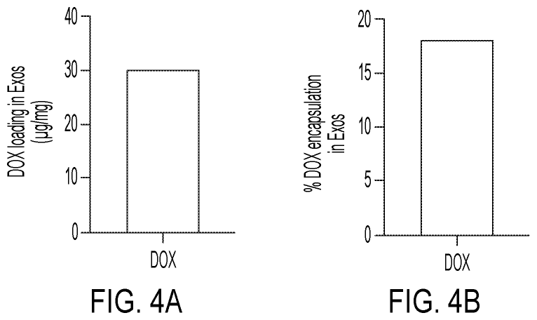

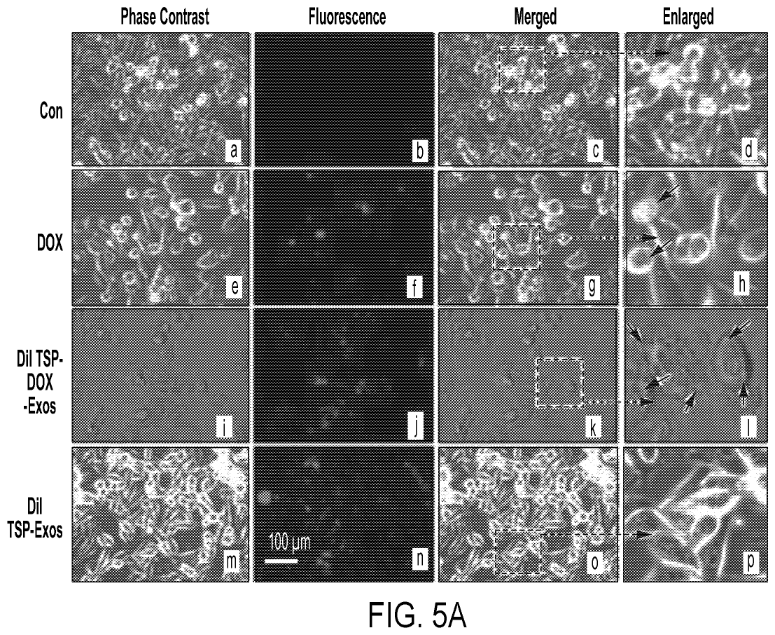

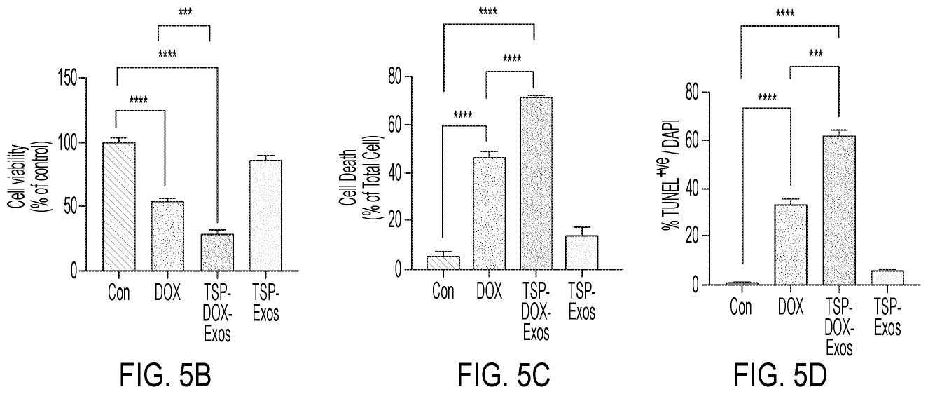

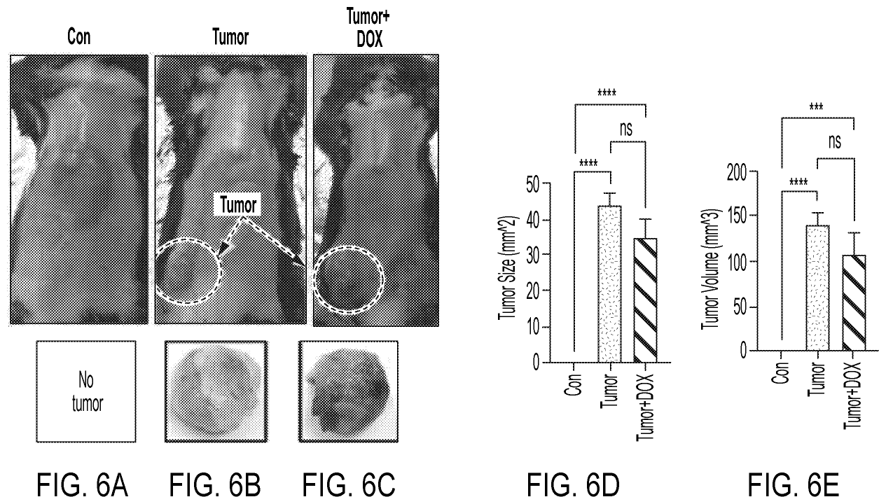

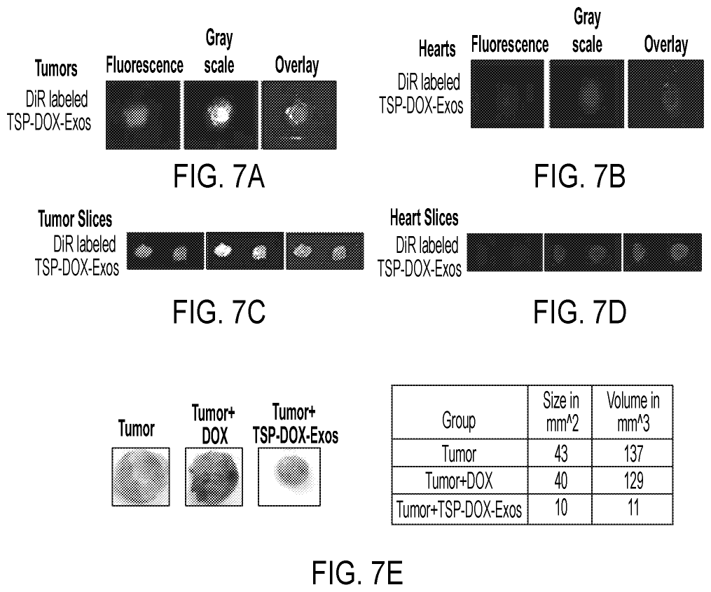

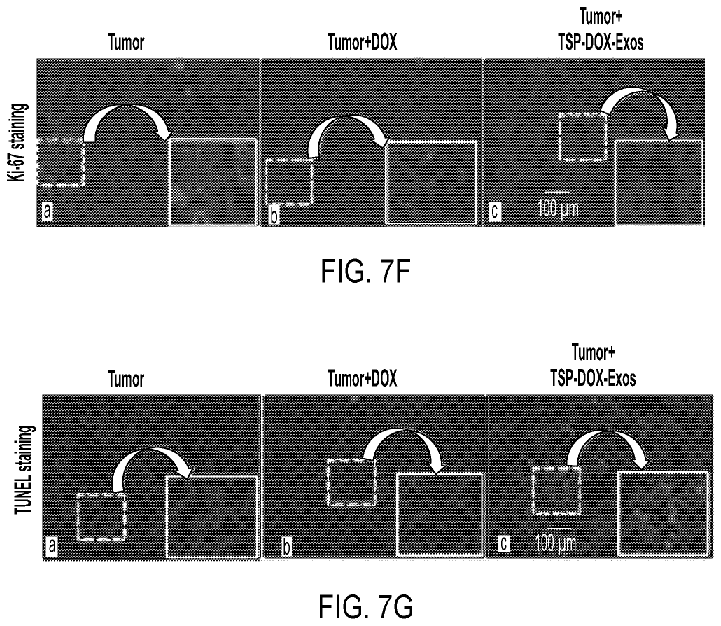

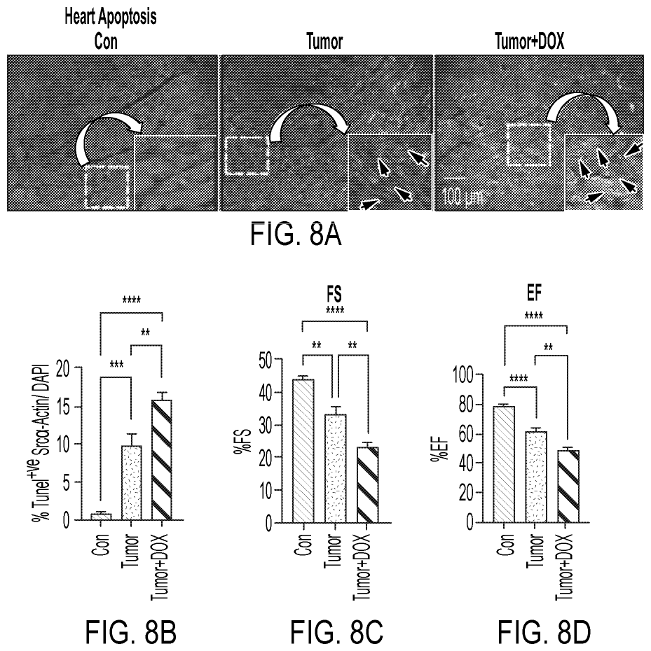

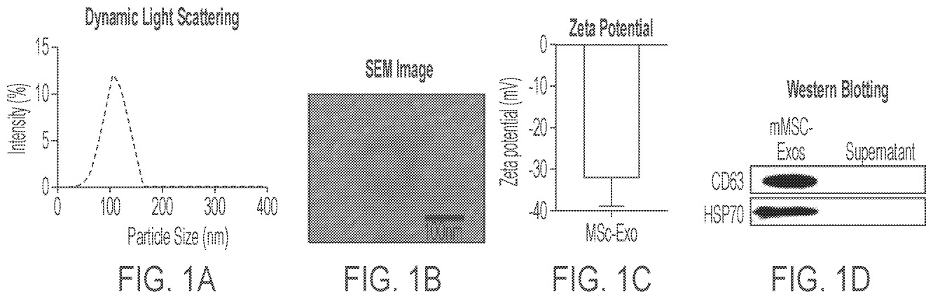

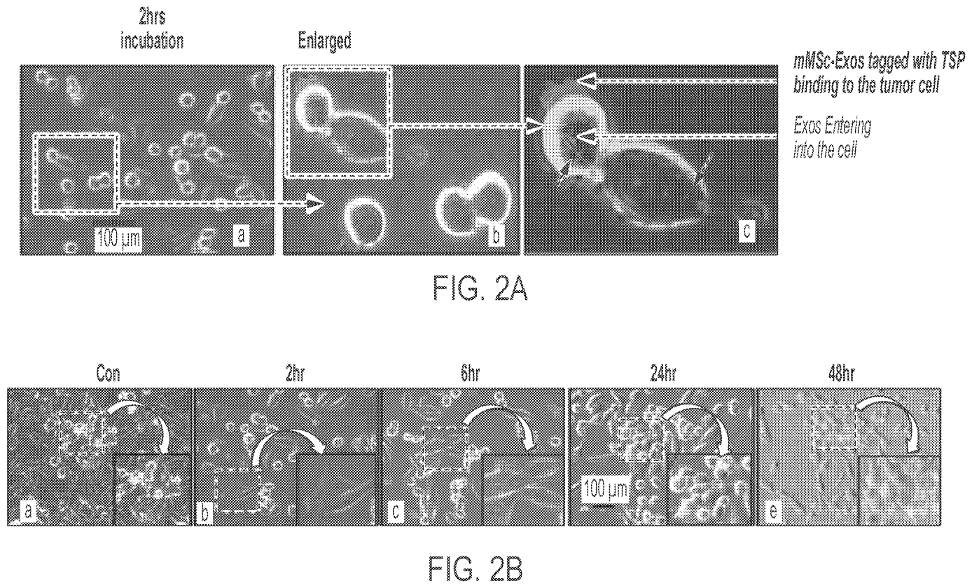

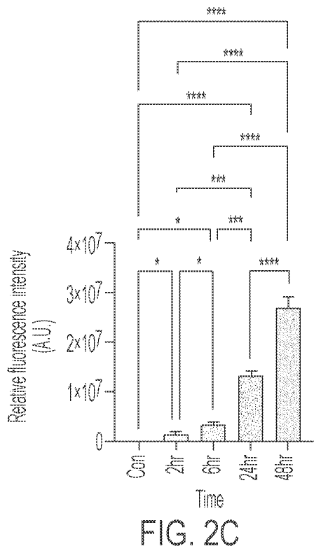

To assess whether the exosome compositions of the present technology specifically target tumor cells, TSP iRGD (SEQ ID NO: 1) was used in the exosome-containing compositions. Exosomes incorporating TSP were labeled with fluorescent dyes Dil (for in vitro studies) and DiR (for in vivo studies). FIG. 2A shows TSP (iRGD)-Exos labeled with fluorescence dye Dil.

### DSPE-PEG5000-N3-iRGD Synthesis

DSPE-PEG5000-N3 was incubated with iRGD peptide (Genscript, USA) in (1:1 ratio) in DMSO using click chemistry. The copper source for the click chemistry reaction was gotten by adding premixed solution of copper (II) sulfate (CuSO4) and stabilizing agent N,N,N′,N′,N″-pentamethyl diethylenetriamine (PMDETA) in a 5:1 molar ratio for 2 hrs. Thereafter, sodium ascorbate prepared in distilled water was added to the reaction mixture. The molar ratio of DSPE-PEG5000-N3:IRGD:CuSO4:ascorbate are in 1:1:0.1:0.2 molar ratio. The solution stirred for 72 hrs at 4 °C. Subsequently, the solution was transferred to a 3.5-5 kDa dialysis bag and dialyzed against water for 72 hrs.

iRGD-DSPE-PEG5000-MSC Exosomes Synthesis

DSPE-PEG500-iRGD was gently mixed with 500 μg of MSC exosomes and incubated at 37 °C. Then, the 100 kda centrifugal filter was used to filter the solution. Next, DSPE-PEG500-iRGD-MSC exosomes were incubated with 5 μM of Dil labeling dye. DSPE-PEG500-iRGD-MSC exosomes were washed using a 100 KDa centrifugal filter to remove unbound exosomes and Dil/DiR dye. Unlabeled as well as Dil/Dir labeled iRGD tagged mMSC exosomes were collected and stored at −80 °C.

### Example 3: Validating TSP-Exo Recognition of TNBC and Cell Penetration

To assess whether TSP-Exos of the present technology may recognize and/or penetrate TNBC cells, TSP-Exos of Example 2 were assessed against E0771 murine TNBC cells and human MDA-MB-231 cancer cells.

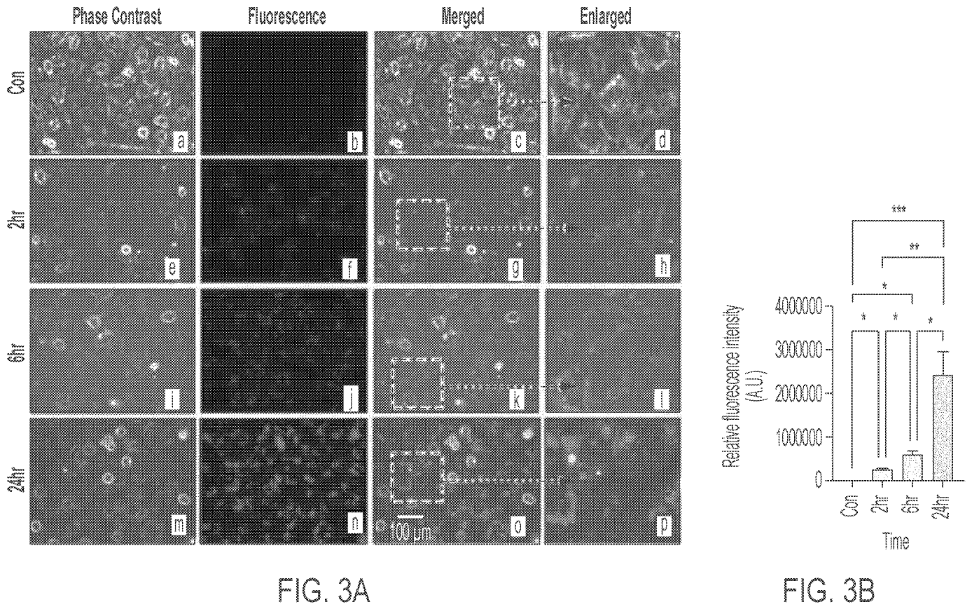

E0771 cells were maintained as a monolayer in culture and were later plated and incubated with Dil labeled TSP-mMSC-Exos for 2-48 hours. Within the first two hours, TSP-exosomes (TSP-Exos) bound TNBC cancer cell surfaces and initiated internalization (FIG. 2A). TSP-Exos internalization increased over time at 6 and 24 hours (FIG. 2B). More than 90% of TNBC cells were positive with TSP-Exos within 48 hours (FIG. 2B). Fluorescence intensity quantification further confirmed increased fluorescence intensity over time (2-48 hours post-treatment) in TNBC cells (FIG. 2C), suggesting TSP-Exos are internalized and present in the cancer cells. Similar data was observed with TNBC human MDA-MB-231 cancer cells (FIGS. 3A and 3B). This indicated that TSP-tagging to MSc exosomes was successful and that TSP-Exos may bind and internalize in TNBC tumor cells of E0771 and MDA-MB-231.

### Example 4: Validation of Doxorubicin (DOX) Loading in TSP-Exos

TSP-Exos (500 μg) of Examples 2 and 3 were mixed with DOX (1 mg/mL) to assess DOX loading in TSP-Exos. Sonication methods were used to encapsulate DOX in TSP-Exos (see Mukhopadhya A Doxorubicin Loading into Milk and Mesenchymal Stem Cells' Extracellular Vesicles as Drug Delivery Vehicles. Pharmaceutics 2023, 15, the disclosure of which is incorporated herein by reference in its entirety). Unbound DOX was removed by filtration. The loading efficiency of DOX was determined by US-visible (UV-Vis) spectrophotometry (excitation; 485 nm, emission: 535 nm) with an established standard curve of DOX concentrations. The drug loading and encapsulation efficiency of the TSP-DOX-Exo drug carrier was calculated using the following formulas:

Drug loading=drug loading in drug carrier Exos (ng)/total mass of drug carrier (mg) encapsulation efficiency=drug loading in drug carrier Exos (ng)/total drug (ng)×100%.

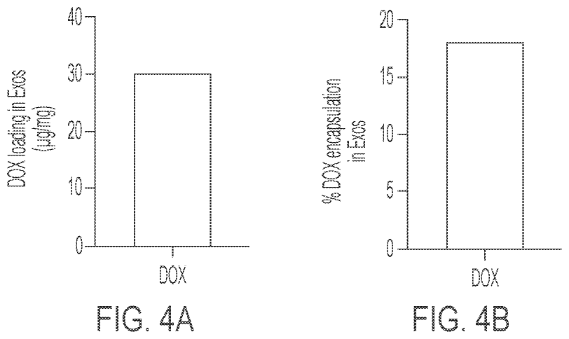

FIG. 4A shows ~28 ng/mg loading of DOX in the TSP-Exos with ~17% efficiency (FIG. 4B) of DOX encapsulation. TSP-DOX-Exos were used for further testing on TNBC tumor cells in vitro and in vivo.

### Example 5: Assessment of TSP-DOX-Exos Against Cancer Cells

To assess cancer-killing abilities of TSP-DOX-Exos, cell cultures were assessed across the following groups: (1) E0771, non-treated controls; (2) E0771+DOX; (3) E0771+TSP-DOX-Exos, test group; and (4) E0771+TSP Exos without DOX. Cells were treated for 24 hours and observed for cell viability, cell death, and TUNEL cell apoptosis.

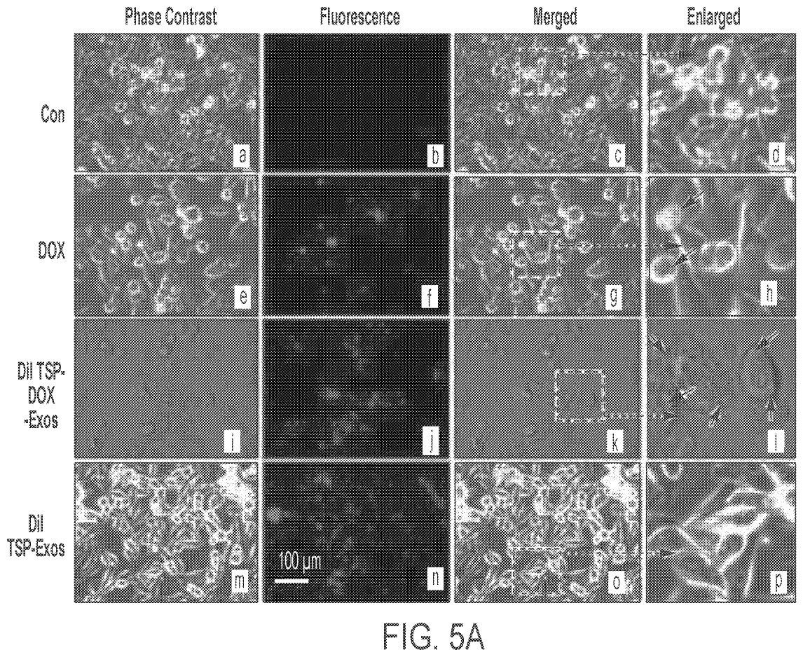

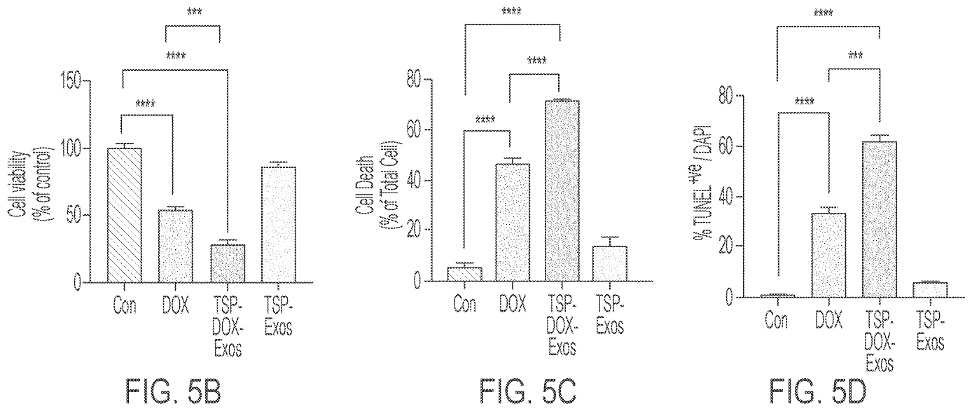

Photomicrograph data (FIG. 5A, i-l) showed that TSP-DOX-Exos of Example 4 had significant binding to cancer cells, with DOX-containing Exos internalization in TNBC cancer cells compared with other groups. Photomicrographs also show decreased cell numbers, as most cancer cells in this group are killed after 24 hours. Next, photomicrograph (FIG. 5A) and quantitative cell viability data show TSP-DOX-Exos treated TNBC cells had significantly reduced cell viability compared with controls (FIG. 5B). The TSP-DOX-Exos group showed distorted morphology and fewer cells due to increased cell death. It was also observed that TSP-DOX-Exos were different from the DOX treatment group (FIG. 5B), suggesting that to inhibit TNBC tumor cell growth, DOX must enter inside the cell, which was well achieved using TSP-DOX-Exos.

Next, the cell death data, as observed using trypan blue staining, suggested an increased cell death in TSP-DOX-Exos compared with normal controls and with a group treated with DOX alone (FIG. 5C). This data was further validated with quantification for apoptosis using TUNEL staining (FIG. 5D). This data set suggests that TSP-DOX-Exos acts as an effective DOX drug carrier and may bind, internalize, and deliver DOX inside the cancer cells to induce cancer cell death and reduce cell viability. In contrast, as observed, free DOX did not enter the cells to kill them efficiently compared with TSP-DOX-Exo (FIGS. 5B-5D).

### Example 6: Assessing TSP-DOX-Exos in TNBC Mouse Models

TSP-DOX-Exos were and will be assessed in the following models: (1) a mouse E0771 tumor model, (2) a human breast cancer metastasizing lung tumor model, and (3) the human patient-derived xenografts (PDX) tumor model for proposed experiments.

### Mouse E0771 Tumor Model

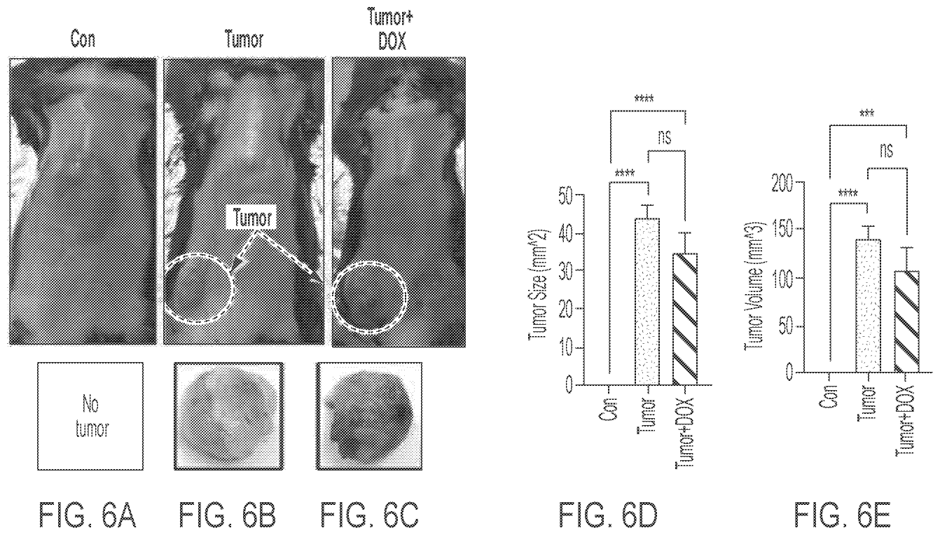

E0771 cells were injected near the fat pad of the fourth mammary gland in the lower abdomen at a density of 2.5×10⁵ cells/200 μl/mouse in C57BL/6J mice. Control mice were injected with PBS. The tumor was grown for 19 days. The 3rd group received DOX on Day 19. After two weeks, post-treatment of DOX and with a total time of 35 days of tumor growth, tumor size and volume were measured, and animals were sacrificed. The photomicrographs (FIGS. 6A and 6B) showed tumor evidence after 35 days, and FIG. 6C shows the marginal decrease in tumor size with DOX treatment. Further, the quantitative data showed that tumor size and volume were not significantly reduced following DOX treatment alone (FIGS. 6D and 6E), suggesting DOX may not penetrate the TNBC tumors.

### Human Metastasizing TNBC Tumor Lung Model

A human triple-negative breast cancer cell MDA-MB-231 variant LM2 will be used in the experimental lung metastatic mouse model. In this study, the role of KLF8-CXCR4 signaling will be tested for the lung metastasis of the cancer cells injected into the tail vein of nude mice. The metastatic growth and progression will be determined using bioluminescent live imaging quantification and analysis, lung whole mount, and immunohistochemical staining of the metastatic nodules. It is proposed to adapt this mouse model for further assessment of the TSP-DOX-Exos.

### Human PDX Model

Tumor tissue fragments from two TNBC PDXs: (1) a TNBC PDX from a patient with Stage IIA/Grade 3 cancer and characterized as an invasive breast ductal carcinoma from a lung metastasis and (2) a TNBC PDX from a patient with Stage IA/Grade 3 cancer and characterized as an invasive breast ductal carcinoma from a primary breast tumor. These PDX tumors will be treated with 2-3 mg/kg DOX intravenously (IV) once weekly over 2-3 weeks exhibited progressive disease, compared with the respective PDX controls treated with 5% dextrose in water 5 ml/kg; IV once per week. Different PDX models will be implanted into the mammary pads of different individual mice to understand the effectiveness of TSP-DOX-Exos to inhibit tumor growth. Remaining tumors from the study will be used to evaluate molecular markers for decreased proliferation (e.g., Ki67), increased apoptosis (e.g., cleaved caspase-3), and/or to understand protein expression changes in treated vs. untreated tumors.

### Example 7. TSP-DOX-Exosomes Penetrate E0771 Tumors and Reduce Tumor Size In Vivo

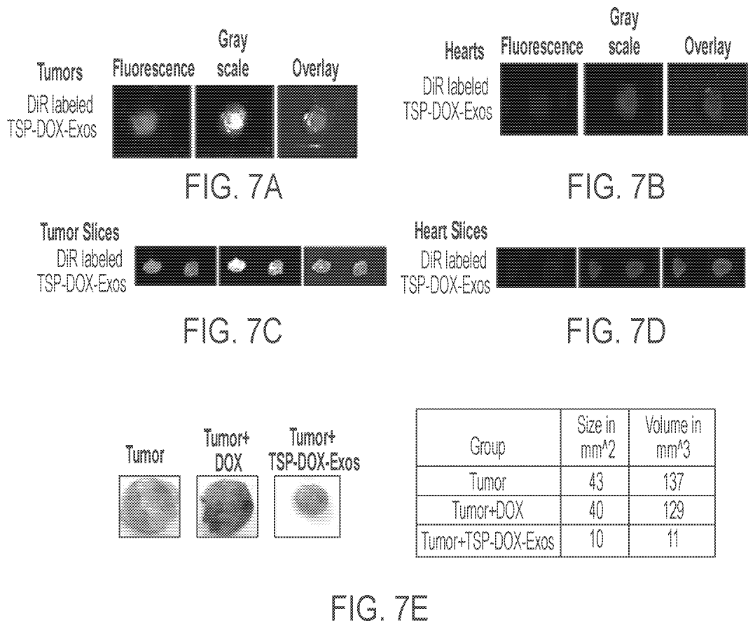

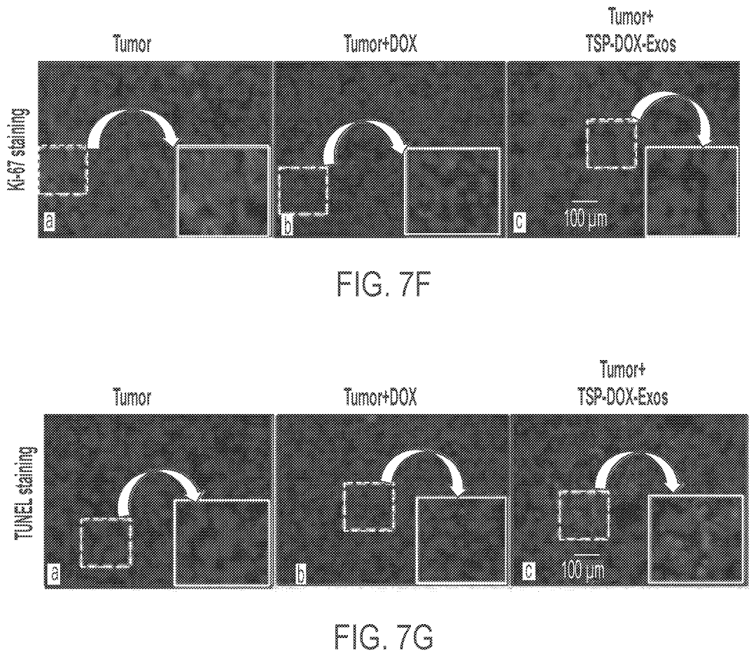

After creating the TNBC E0771 tumor model as mentioned above, it was investigated whether TSP-DOX-Exos target tumors in vivo and their effects on tumor size and proliferation. Using the tumor model as discussed above, after 19 days of tumor growth, TSP-DOX-Exos was injected and DOX as a control. Animals were examined after seven days to establish whether TSP-DOX-Exos traveled to the TNBC tumors and/or to heart, as this is another focus of the current investigation. The data shows that TSP-DOX-Exos were localized in the tumors (FIG. 7A) but not in the heart (FIG. 7B). Further, tumor tissue sample were sliced to confirm their penetration inside the tumors, and the data shows these exosomes penetrated deep inside the tumors (FIG. 7C) but not in the hearts (FIG. 7D).

Next, TSP-DOX-Exos' effects on tumor size and volume were observed. The tumor image data shows that tumor size and volume were reduced in the TSP-DOX-Exos group (FIG. 7E) following one week of treatment compared with the DOX group. It is essential to investigate whether DOX-Exos that penetrate the cell affect cancer cell proliferation; Ki67 data shows TSP-DOX-Exos significantly decreases cancer cell proliferation (FIG. 7F) compared with tumor controls and animals treated with DOX, which is very encouraging. Further, this data shows increased apoptosis in the TSP-DOX-Exos-treated group compared with controls (FIG. 7G) (confirmed by TUNEL staining). This suggests that exosome compositions of the present technology comprising DOX may penetrate the cancer cells to kill TNBC tumors

### Example 8. Assessment of DOX-Induced Cardiomyopathy in Mouse Tumor Models

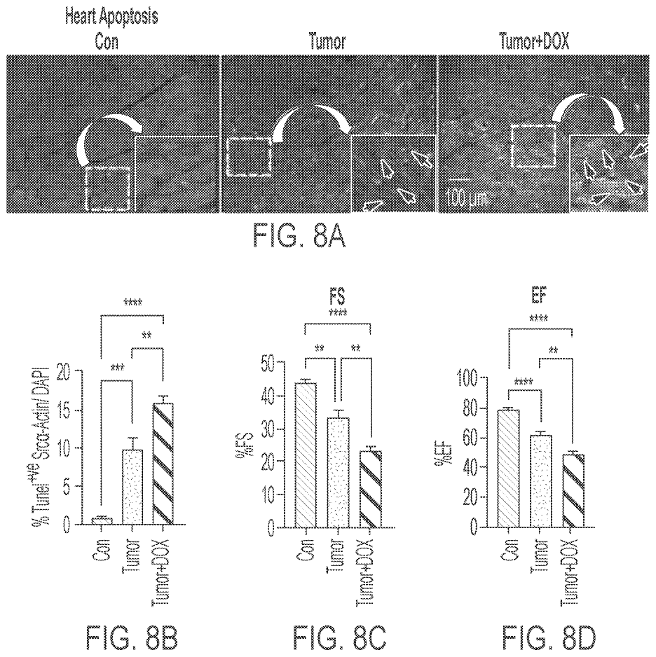

To assess DOX-treatment effects on cardiomyopathy in mice, control, tumor, and tumor+ DOX-treated groups were used to investigate the presence of apoptosis and cardiac function. The data showed significantly increased apoptosis (FIG. 8A) in the tumor and tumor+ DOX group compared with controls. Quantitative apoptosis data validated this data (FIG. 8B). The echocardiography data confirmed reduced cardiac function (FIGS. 8C and 8D) in tumor-bearing animals. DOX treatment was used to reduce tumors, which further reduced cardiac function, which is significant compared with non-treated tumors (FIGS. 8C and 8D). This data set confirmed a tumor-bearing animal with DOX that induces cardiotoxicity were successfully created. TSP-DOX-Exos injected in tumor-bearing animals targeted tumors, and did not impact the heart (FIGS. 7A and 7B).

### Example 9. Identification, Characterization, and Validation of DOX Encapsulated in Mouse and Human TSP-MSc-Exos

### Creation of Human TSP-DOX-Exos, Binding to Cancer Cells, and DOX Loading by Sonication Method:

Bone marrow-human mesenchymal stem cells (hMScs) were cultured using a complete growth medium kit. Human blood samples were obtained and human blood MSC-Exos were isolated. hMSc was cultured and used to extract hMSc-Exos (see Shu S et al., A Rapid Exosome Isolation Using Ultrafiltration and Size Exclusion Chromatography (REIUS) Method for Exosome Isolation from Melanoma Cell Lines. Methods Mol Biol 2021, 2265:289-304, the disclosure of which is incorporated herein by reference in its entirety).

Using size exclusion chromatography (SEC), exosomes were isolated and characterized using DLS, SEM, zeta potential, and western blotting methods, as was described for mouse exosomes. Mouse and human Exos were also characterized for additional markers (CD9, CD81, TSG101) using ELISA and western blotting.

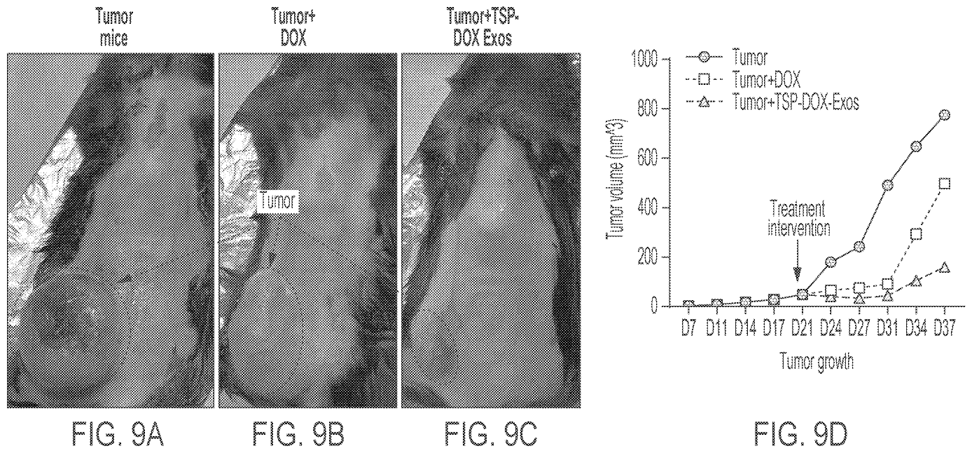

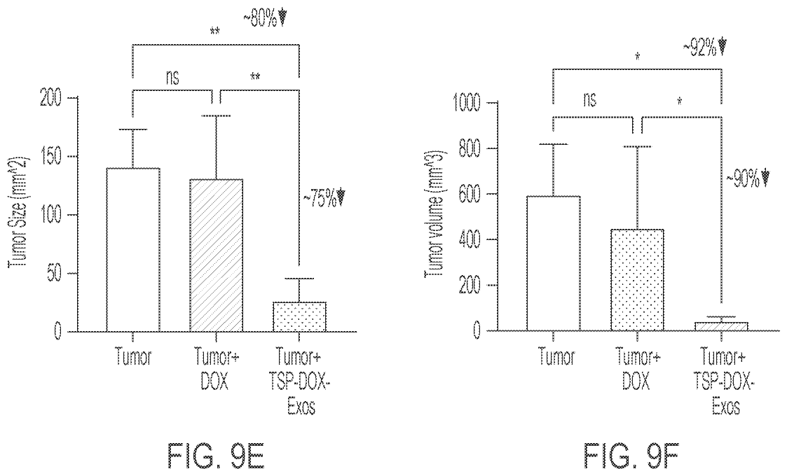

hMSC-Exos were tagged with TSP (iRGD; SEQ ID NO: 1) and loaded with DOX and similarly assessed as outlined in Examples 2 and 4 (TSP-DOX-Exos). Efficacy against cancer cells was similarly assessed as outlined in Examples 5. Like the earlier animal studies, long term animal studies showed a reduction in TNBC tumor size and volume in TSP-DOX-Exo treated mice compared to controls, including those treated with DOX alone (FIGS. 9A-9F). These effects were observed through day 37, with dosing occurring at day 31.

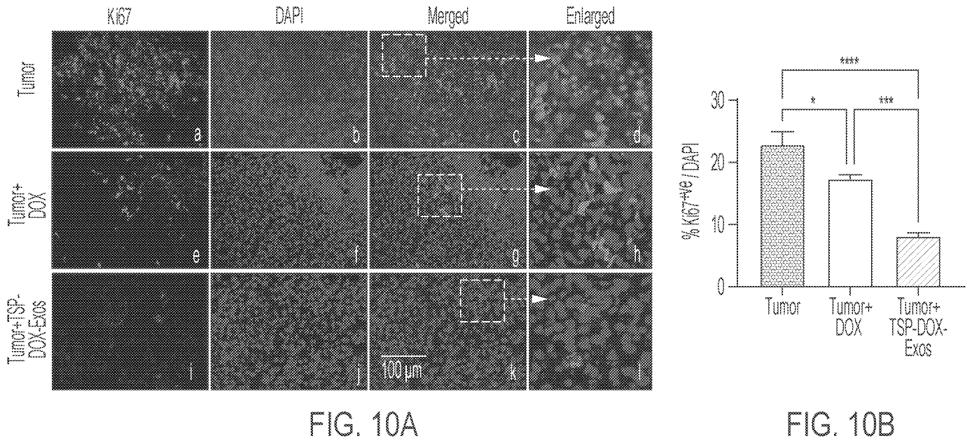

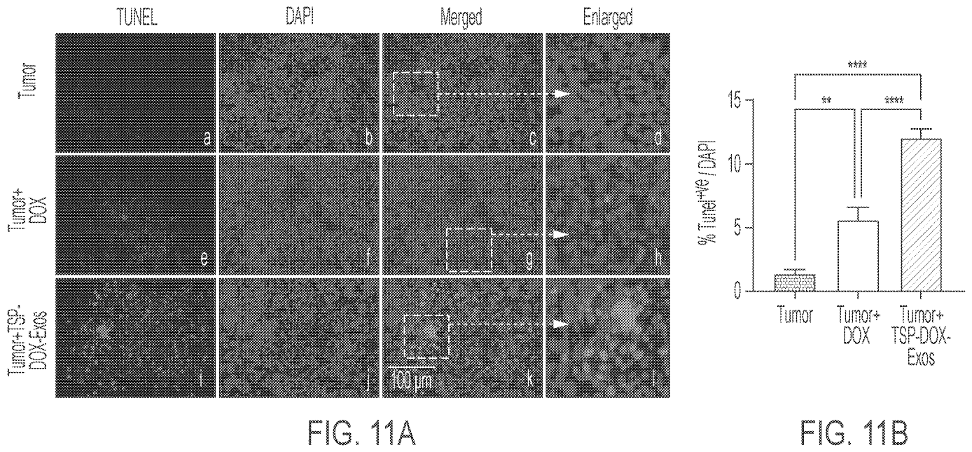

On a cellular level, TSP-DOX-Exos also showed evidence of TNBC cell reduction, including reducing growth, reducing viability, and increasing death of TNBC cancer cells, as validated with Ki-67 and TUNEL staining (FIGS. 10A, 10B, 11A, and 11B).

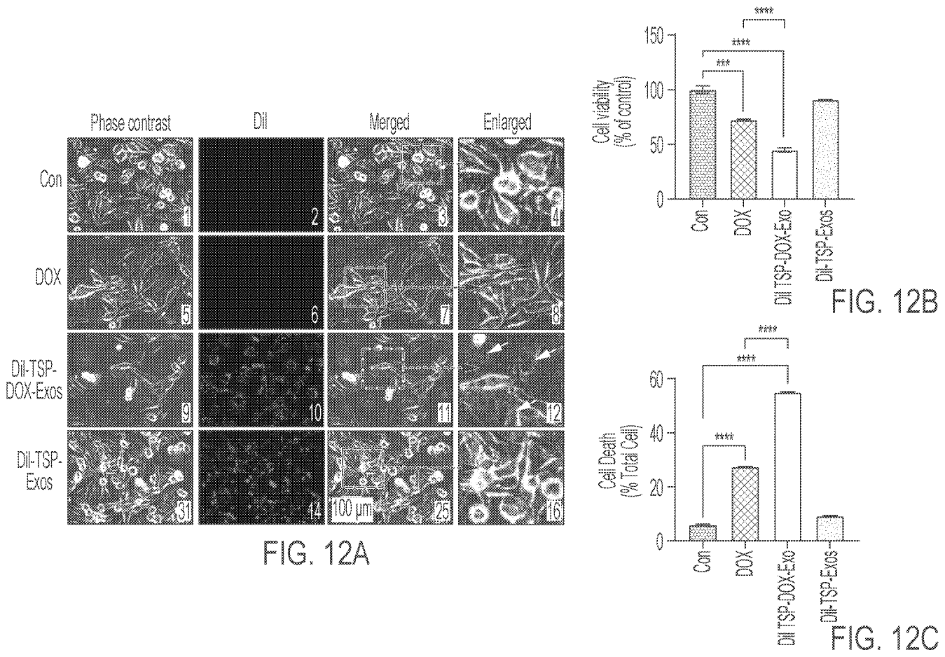

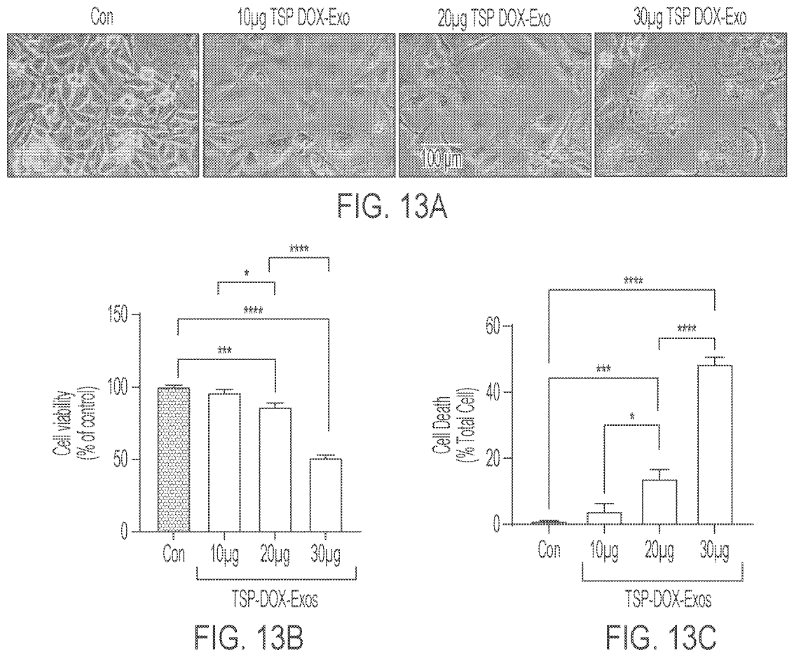

When assessing TSP-DOX-Exos against human TNBC (hTNBC) cells in culture, it was demonstrated that TSP-DOX-Exos showed increased killing of cancer cells, as assessed by a reduction in cell viability and an increase in cell death measurements, relative to controls (FIGS. 12A-12C). To examine these outcomes under various TSP-DOX-Exos concentrations, cell viability and cell death were assessed in hTNBC cultured cells that were either untreated or treated with 10 μg, 20 μg, or 30 μg of TSP-DOX-Exos (FIGS. 13A-13C). This demonstrated that increasing doses of TSP-DOX-Exos reduced hTNBC cell viability and increased hTNBC cell death, relative to controls.

### Example 10. Determining the Therapeutic Potential of Novel TSP (iRGD)-DOX-Exos

To test the hypothesis that the mouse TSP-DOX-Exos treatment enhanced apoptosis of TNBC tumors, resulting in reduced tumor growth and volume, the following groups were assessed: (1) E0771 TNBC mouse model, (2) mouse model of M2 lung metastasis, and (3) human PDX mouse model.

To investigate whether treatment with TSP-DOX-Exos inhibited tumor size and volume in E0771 TNBC tumors: a C57BL/6J syngeneic tumor mice model of mammary adenocarcinoma was developed using E0771 cells using a published protocol (see Ewens A et al., Distant metastasis from subcutaneously grown E0771 medullary breast adenocarcinoma. Anticancer Res 2005, 25:3905-15, the disclosure of which is incorporated herein by reference in its entirety).

C57BL/6J female mice of age 10±2 weeks were injected with E0771 cells near the fat pad of the fourth mammary gland in the lower abdomen. Animals were allowed to grow the tumor for 19 days. Tumor size was examined, and treatments started after tumors became ~0.5 cm×0.5 cm in surface area (day 0).

Animals were divided into four groups: (1) Tumor alone without treatment, (2) Tumor+DOX, (3) Tumor+TSP-Exos, and (4) Tumor+TSP-DOX-Exos. DOX was injected in three intraperitoneal (i.p) injections (4 mg/kg body weight), on alternative days with a cumulative dose of 12 mg/kg. The TSP-Exos dose was 50 μg (i.v.) of exosome protein concentration with a cumulative dosage of 150 μg in three days. This 50 μg of exosome protein contained ~0.6 μg of DOX (encapsulated). Three doses of TSP-DOX-Exos were given in three alternate days with a cumulative dose of 1.8 μg of DOX. This encapsulated DOX dosage was ~160-fold less than the free DOX (12 mg/kg) used to treat tumors.

As shown in the previous working examples, FIGS. 7A-7E show that TSP-DOX-Exos was adequate as it reduced tumor size and volume, suggesting that TSP-DOX-Exos delivered DOX inside the tumors. Tumor survival was monitored and recorded weekly. Tumor volumes were calculated by the following formula: (½×L×W×H), in which L was the length, W was the width, and H was the height, as shown in FIGS. 6A-6E.

Animals will be investigated at 5-, 8- and 10 weeks post-treatment to track DIR labeled exosomes to determine the location of fluorescence labeled exosomes. Blood samples will be collected by heart puncture via exsanguination. Heart, lungs, liver, kidney, spleen, and brain will be collected, washed with phosphate buffer saline (PBS), and further imaged to track if TSP-DOX-Exos has been housed in other organs. Tumor cell proliferation and apoptosis will be investigated. Based on power analysis, 14-16 female mice are needed per group to observe the desired coefficient of variation (cv) with a range of 0.4 to 1.0. Thus, for a cv of 30%, a power of 80% is needed to detect 26% changes at a 0.05 significance level.

### Assessing Whether TSP-DOX-Exos Reduces Cell Proliferation and Increases Apoptosis in Tumors

Tumor paraffin sections for the above mice will be prepared. Immunohistochemistry (IHC) staining for Ki67 will be performed using standard protocols. Tumor sections will be deparaffinized, rehydrated and blocking will be performed with 10% goat serum and will be stained with Ki67 primary antibody overnight at 4 °C. Quantitative data for cell proliferation will be calculated by dividing positive cells over total DAPI times 100 [(total cells+ve/total DAPI)*100], and GraphPad Prism 10 software version will be used for graphical representation. Apoptosis will be examined using TUNEL staining. Apoptosis will also be confirmed using western blotting and RT-PCR for BAX/Bcl2 and caspase-3.

These experiments will be repeated to assess efficacy of TSP-DOX-Exos treatment on M2 lung metastasis. To test the hypothesis that TSP-DOX-Exos-guided targeted chemotherapy is effective in inhibiting TNBC metastatic growth, the MDA-MB-231 human TNBC variant M292-94 is injected into the tail vein of female immunocompromised mice.

Additionally, it will be assessed whether TSP-DOX-Exos treatment may effectively inhibit human PDXs from primary and metastatic patients implanted in mice's mammary tissue. Tumor tissue fragments from the human PDX model of Example 6 will be assessed Tumors will be were collected when in active growth phase but before necrosis. In brief, tumors will be harvested, washed, and minced into fragments of ~2×2×3 mm for implantation into the mammary pad of NSG mice. As outlined in the prior examples, treatment groups will include control vehicle, TSP-Exos, TSP-DOX-Exos, and DOX alone and will be assessed as outlined in examples 5-9. Tumor microenvironment and spreading will be evaluated for staining with markers such as cytokeratin AE1AE3.

### Example 11. Assessing Whether TSP-DOX-Exos Reduce Cardiotoxicity without Interfering with its Anti-Tumor Properties

To confirm that TSP-DOX-Exos will not induce cardiotoxicity but enhance anti-tumor properties for TNBC tumors, three different tumor models will be assessed: (1) the E0771 TNBC mouse model, (2) the mouse model of metastatic human TNBC, and (3) the human PDX mouse model of the prior examples. Effects of TSP-DOX-Exos on anti-tumor properties will be compared with other groups.

### Cardiac Function

2D echocardiography will performed and M-mode images from at least three consecutive cardiac cycles of the left ventricle (LV) will be captured and analyzed. The left ventricular internal dimension-diastole (LVIDd), left ventricular internal dimension-systole (LVIDs), and LV end-diastolic and systolic volumes (EDV and ESV, respectively) will be calculated. Indices of LV systolic functions including fractional shortening [FS; (LVIDd−LVIDs)/LVIDd×100)], and ejection fraction [EF; (EDV-ESV)/EDV×100)] will be determined. Further, M-mode echocardiography images in a short axis view will be calculated using LV mass, LV mass to body ratio, heart failure preserved ejection fraction (HFpEF) HF reduced EF (HFrEF), and fractional shortening (FS).

### Assessment of Cardiac Remodeling and Apoptosis

DOX-induced cardiomyopathy on cardiac fibrosis, cytoplasmic vacuolization, and apoptosis will be assessed. Tissue sections will be analyzed, and cardiac hypertrophy will be evaluated using RT-PCR and western blotting by hypertrophy genes ANP, BNP, and MyH7. To examine apoptosis, TUNEL co-staining with sarcomeric α-actin, will be used to assess cardiac myocytes apoptosis. Also, staining for pro-apoptotic proteins caspase-3/Bax will be performed. Heart sections are counterstained with DAPI to show total nuclei.

### Additional Embodiments

Various embodiments of the present technology are set forth below in paragraphs to [0305]:
1. An exosome composition comprising or consisting of an exosome at least partially conjugated to one or more tumor-specific peptides (TSPs) that bind at least a portion of a cancer cell or a tumor microenvironment.
2. The exosome composition of embodiment 1, further comprising one or more anticancer agents at least partially encapsulated within the exosome.
3. The exosome composition of embodiment 1 or 2, further comprising one or more anticancer agents at least partially encapsulated within the exosome.
4. An exosome composition comprising or consisting of (i) an exosome at least partially conjugated to one or more TSPs that bind at least a portion of a cancer cell or a tumor microenvironment, and (ii) one or more anticancer agents at least partially encapsulated within the exosome.
5. A method of targeting a cancer in a subject in need thereof relative to a control, the method comprising administering to the subject:
an exosome composition comprising or consisting of (i) an exosome at least partially conjugated to one or more TSPs that bind at least a portion of a cancer cell or a tumor microenvironment, and (ii) one or more anticancer agents at least partially encapsulated within the exosome.
6. The method of embodiment 1, wherein the cancer is triple negative breast cancer (TNBC).
7. The method of embodiment 5, wherein the cancer is selected from the group consisting of breast cancer, lung cancer, colorectal cancer, prostate cancer, pancreatic cancer, ovarian cancer, cervical cancer, endometrial cancer, liver cancer, gastric cancer, esophageal cancer, kidney cancer, bladder cancer, melanoma, thyroid cancer, head and neck cancer, brain cancer, testicular cancer, sarcoma, bone cancer, lymphoma, multiple myeloma, and leukemia.
8. A method of enhancing one or more anticancer agent effects in a subject in need thereof, relative to a control, the method comprising administering to the subject:
an exosome composition comprising or consisting of (i) an exosome at least partially conjugated to one or more TSPs that bind at least a portion of a cancer cell or a tumor microenvironment, and (ii) one or more anticancer agents at least partially encapsulated within the exosome.
9. The method of embodiment 8, wherein the one or more anticancer agent effects is selecting from the group consisting of reducing or preventing an increase in a tumor size, reducing or preventing an increase in a tumor volume, and/or reducing or preventing a cancer metastasis level in a subject in need thereof, relative to a control.
10. A method of reducing or preventing an anticancer agent toxicity level in a subject in need thereof, relative to a control, the method comprising administering to the subject:
an exosome composition comprising or consisting of (i) an exosome at least partially conjugated to one or more TSPs that bind at least a portion of a cancer cell or a tumor microenvironment, and (ii) one or more anticancer agents at least partially encapsulated within the exosome.
11. The method of embodiment 10, wherein the anticancer agent toxicity comprises anticancer agent-induced cardiomyopathy or cell death in non-cancer cell populations.
12. The method of embodiment 11, wherein the anticancer agent-induced cardiomyopathy is doxorubicin (DOX)-induced cardiomyopathy.
13. The method of embodiment 11 or 12, wherein the method reduces one or more of a cardiac fibrosis level, a cytoplasmic vacuolization level, or a cardiac apoptosis level, relative to a control.
14. The exosome composition or the method of any one of embodiments 3 to 13, wherein the one or more anticancer agents is selected from the group consisting of an alkylating agent, a platinum agent, an antimetabolite, a topoisomerase inhibitor, a microtubule-targeting agent, and a cytotoxic agent.
15. The exosome composition or the method of embodiment 14, wherein the alkylating agent is selected from the group consisting of cyclophosphamide, ifosfamide, melphalan, and bendamustine.
16. The exosome composition or the method of embodiment 14, wherein the platinum agent is selected from the group consisting of cisplatin, carboplatin, and oxaliplatin.
17. The exosome composition or the method of embodiment 14, wherein the antimetabolite is selected from the group consisting of 5-fluorouracil (5-FU), capecitabine, methotrexate, cytarabine, gemcitabine, and pemetrexed.
18. The exosome composition or the method of embodiment 14, wherein the topoisomerase inhibitor is selected from the group consisting of doxorubicin (DOX), epirubicin, irinotecan, topotecan, etoposide, and teniposide.
19. The exosome composition or the method of embodiment 14, wherein the microtubule-targeting agent is selected from the group consisting of paclitaxel, docetaxel, vincristine, vinblastine, vinorelbine, and cabazitaxel.
20. The exosome composition or the method of embodiment 14, wherein the cytotoxic agent is selected from the group consisting of bleomycin, dacarbazine, and temozolomide.
21. The exosome composition or the method of any one of embodiments 1 to 20, wherein the exosome composition is at least partially internalized by the cancer cell.
22. The exosome composition or the method of any one of embodiments 1 to 21, wherein the portion of the cancer cell or the tumor microenvironment comprises a cell surface protein.
23. The exosome composition or the method of embodiment 22, wherein the cell surface protein is a transmembrane protein.
24. The exosome composition or the method of embodiment 23, wherein the transmembrane protein is an integrin or a subunit thereof.
25. The exosome composition or the method of embodiment 24, wherein the integrin is selected from the group consisting of an arginine-glycine-aspartic acid (RGD)-binding integrin, a collagen-binding integrin, a laminin-binding integrin, and a leukocyte-specific integrin.
26. The exosome composition or the method of any one of embodiments 1 to 25, wherein the one or more TSPs comprise an RGD motif.
27. The exosome composition or the method of embodiment 26, wherein the one or more TSPs comprise or consist of an amino acid sequence at least about 100%, 99%, 98%, 95%, 90%, 85%, 80%, 75%, 70%, 65%, 60%, or 50% identical to the amino acid sequence of CRGDKGPDC (SEQ ID NO: 1).
28. An exosome composition comprising or consisting of (i) an exosome at least partially conjugated to one or more TSPs comprising or consisting of an amino acid sequence of CRGDKGPDC (SEQ ID NO: 1) that at least partially bind a cell surface integrin of a cancer cell, and (ii) DOX at least partially encapsulated within the exosome.
29. The exosome composition or the method of any one of embodiments 24, 25, or 28, wherein the integrin is selected from the group consisting of αvβ1, αvβ3, αvβ5, αvβ6, αvβ8, α8β1, αIIbβ3, α1β1, α2β1, α3β1, α10β1, α11β1, α6β1, α7β1, α6β4, αLβ2, αMβ2, αXβ2, αDβ2, α4β1, α4β7, α9β1, and αEβ7.
30. The exosome composition or the method of any one of embodiments 1 to 29, wherein the exosome is at least partially conjugated to the one or more TSPs by a lipid-polymer conjugate.
31. The exosome composition or the method of embodiment 30, wherein the lipid-polymer conjugate comprises or consists of one or more of a phospholipid, a polymer, and a functional group.
32. The exosome composition or the method of embodiment 31, wherein (i) the phospholipid is 1,2-distearoyl-sn-glycero-3-phosphoethanolamine (DSPE); (ii) the polymer is poly(ethylene glycol) (PEG5000); and/or (iii) the functional group is an azide functional group.
33. The exosome composition or the method of any one of embodiments 1 to 32, wherein the exosome is a mesenchymal stem cell-derived exosomes (MSc-exosome).
34. The exosome composition or the method of any one of embodiments 1 to 32, wherein the exosome is selected from the group consisting of an adipose-derived stem cell exosome (asc-exosome), a bone marrow-derived mesenchymal stromal/stem cell exosome (bmsc-exosome), an umbilical cord mesenchymal stromal/stem cell exosome (ucmsc-exosome), a dendritic cell-derived exosome (dc-exosome), a T cell-derived exosome, a natural killer cell-derived exosome (NK cell-derived exosome), a B cell-derived exosome, a macrophage-derived exosome, an M1 macrophage-derived exosome (M1-exosome), an M2 macrophage-derived exosome (m2-exosome), a platelet-derived exosome, an erythrocyte-derived exosome, an endothelial cell-derived exosome, a fibroblast-derived exosome, a neural stem cell-derived exosome, a neuron-derived exosome, an astrocyte-derived exosome, a microglia-derived exosome, a cardiomyocyte-derived exosome, a hepatocyte-derived exosome, a tumor-derived exosome (tdex), a cancer-associated fibroblast-derived exosome (caf-derived exosome), a serum-derived exosome, a plasma-derived exosome, a urine-derived exosome, a saliva-derived exosome, a breast milk-derived exosome, an ascites-derived exosome, and a cerebrospinal fluid-derived exosome (csf-derived exosome).
35. The exosome composition or the method of any one of embodiments 1 to 34, wherein the exosome comprises a particle size of about 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, or 200 nm.
36. The exosome composition or the method of any one of embodiments 1 to 35, wherein the exosome comprises a zeta potential of about −100 mV, −90 mV, −80 mV, −75 mV, −70 mV, −60 mV, −65 mV, −50 mV, −45 mV, −40 mV, −35 mV, −30 mV, −20 mV, or −15 mV.
37. The exosome composition or the method of any one of embodiments 1 to 36, comprising an exosome:TSP weight ratio of at least about 1:2500, 1:3000, 1:4000, 1:5000, 1:5500, 1:6000, 1:6500, 1:7000, 1:8000, 1:9000, or 1:10000.
38. The exosome composition or the method of any one of embodiments 1 to 36, comprising an exosome:TSP weight ratio of about 1:6000
39. The exosome composition or the method of any one of embodiments 2 to 27 or 29 to 38, wherein the one or more anticancer agents are loaded into the exosome compositions with a weight ratio of TSP and exosomes:anticancer agent of about 10:1, 11:1, 12:1, 13:1, 14:1, 15:1, 16:1, 17:1, 18:1, 19:1, or 20:1.
40. The exosome composition or the method of any one of embodiments 2 to 27 or 29 to 39, wherein the one or more anticancer agents comprise or consist of two or more anticancer agents.
41. The exosome composition or the method of any one of embodiments 2 to 27 or 29 to 40, wherein the one or more anticancer agents are present in the exosome in an amount of about 10 ng/ml, 20 ng/mL, 50 ng/ml, 100 ng/ml, 200 ng/ml, 500 ng/ml, 1 μg/mL, 2 μg/mL, 5 μg/mL, 10 μg/mL, 20 μg/mL, 50 g/mL, 100 μg/mL, 200 μg/mL, 500 μg/mL, 1 mg/mL, 1.5 mg/mL, 2 mg/ml, 3 mg/mL, 5 mg/mL, 10 mg/mL, 15 mg/mL, or 20 mg/mL.
42. The exosome composition or the method of embodiment 2, wherein the DOX is loaded into the exosome compositions at a weight ratio of TSP and exosomes:DOX of about 14:1.
43. The exosome composition or the method of any one of embodiments 1 to 42, wherein the cancer cell is selected from the group consisting of a breast cancer cell, a lung cancer cell, a colorectal cancer cell, a prostate cancer cell, a pancreatic cancer cell, an ovarian cancer cell, a cervical cancer cell, an endometrial cancer cell, a liver cancer cell, a gastric cancer cell, an esophageal cancer cell, a kidney cancer cell, a bladder cancer cell, a melanoma cell, a thyroid cancer cell, a head and neck cancer cell, a brain cancer cell, a testicular cancer cell, a sarcoma cell, a bone cancer cell, a lymphoma cell, a multiple myeloma cell, and a leukemia cell.
44. The exosome composition or the method of any one of embodiments 1 to 42, wherein the cancer cell is a triple negative breast cancer (TNBC cell).
45. The method of any one of embodiments 5 to 27 or 29 to 44, wherein the subject has or is at risk of developing TNBC.
46. The method of any one of embodiments 5 to 27 or 29 to 45, wherein the subject has or is at risk of developing primary breast cancer or metastatic breast cancer.
47. The method of any one of embodiments 5 to 27 or 29 to 46, wherein the subject has received or is receiving an anticancer agent or a cancer treatment.
48. The method of any one of embodiments 5 to 27 or 29 to 46, wherein the subject has not received or an anticancer agent or a cancer treatment.
49. The method of embodiment 47 or 48, wherein the cancer treatment is selected from the group consisting of an immunotherapy, a radiation therapy, and a surgical therapy.
50. The exosome composition or the method of any one of embodiments 1 to 49, wherein the exosome composition is formulated in a pharmaceutical composition.
51. The exosome composition or the method of embodiment 50, wherein the pharmaceutical composition further comprises one or more pharmaceutically acceptable carriers or excipients.
52. The exosome composition or the method of embodiment 50 or 51, wherein the pharmaceutical composition is formulated for oral, rectal, vaginal, ocular, intranasal, topical, parenteral, injectable delivery, or from a surgical device.
53. The method of any one of embodiments 50 to 52, wherein the pharmaceutical composition is administered to the subject orally, rectally, vaginally, ocularly, intranasally, topically, parenterally, by injection, or form a surgical device.
54. The exosome composition or the method of embodiment 52 or 53, wherein the injectable delivery comprises intravenous (IV), subcutaneous (SC), intramuscular (IM), intraperitoneal (IP), or intracerebroventricular (ICV) injection.
55. The method of any one of embodiments 5 to 27 or 29 to 54, wherein the exosome composition is administered to the subject in an amount of about 1.5 mg/kg/day to about 45 mg/kg/day.
56. The method of any one of embodiments 5 to 27 or 29 to 55, wherein the exosome composition is administered to the subject in an amount of about 0.01 mg/kg/day to about 10.0 mg/kg/day, of the one or more anticancer agents.

From the foregoing, it will be appreciated that specific embodiments of the present technology have been described herein for purposes of illustration, but that various modifications may be made without deviating from the scope of the present technology. Accordingly, the present technology is not limited except as by the appended claims.

I/We claim:

1. An exosome composition comprising or consisting of an exosome at least partially conjugated to one or more tumor-specific peptides (TSPs) that bind at least a portion of a cancer cell or a tumor microenvironment.

2. An exosome composition comprising or consisting of (i) an exosome at least partially conjugated to one or more TSPs that bind at least a portion of a cancer cell or a tumor microenvironment, and (ii) one or more anticancer agents at least partially encapsulated within the exosome.

3. A method of targeting a cancer in a subject in need thereof relative to a control, the method comprising administering to the subject:
an exosome composition comprising or consisting of (i) an exosome at least partially conjugated to one or more TSPs that bind at least a portion of a cancer cell or a tumor microenvironment, and (ii) one or more anticancer agents at least partially encapsulated within the exosome.

4. The exosome composition of claim 2, wherein the cancer cell is triple negative breast cancer (TNBC) cell.

5. The exosome composition of claim 4, wherein the cancer cell is a cell from a cancer selected from the group consisting of breast cancer, lung cancer, colorectal cancer, prostate cancer, pancreatic cancer, ovarian cancer, cervical cancer, endometrial cancer, liver cancer, gastric cancer, esophageal cancer, kidney cancer, bladder cancer, melanoma, thyroid cancer, head and neck cancer, brain cancer, testicular cancer, sarcoma, bone cancer, lymphoma, multiple myeloma, and leukemia.

6. A method of (a) enhancing one or more anticancer agent effects or (b) reducing or preventing an anticancer agent toxicity in a subject in need thereof, relative to a control, the method comprising administering to the subject:
an exosome composition comprising or consisting of (i) an exosome at least partially conjugated to one or more TSPs that bind at least a portion of a cancer cell or a tumor microenvironment, and (ii) one or more anticancer agents at least partially encapsulated within the exosome.

7. The method of claim 6, wherein (a) the one or more anticancer agent effects is selecting from the group consisting of reducing or preventing an increase in a tumor size, reducing or preventing an increase in a tumor volume, and/or reducing or preventing a cancer metastasis level in a subject in need thereof, relative to a control, and (b) the anticancer agent toxicity comprises anticancer agent-induced cardiomyopathy or cell death in non-cancer cell populations

8. The method of claim 7, wherein the anticancer agent-induced cardiomyopathy is doxorubicin (DOX)-induced cardiomyopathy.

9. The method of claim 8, wherein the method reduces one or more of a cardiac fibrosis level, a cytoplasmic vacuolization level, or a cardiac apoptosis level, relative to a control.

10. The exosome composition of claim 2, wherein the one or more anticancer agents is selected from the group consisting of an alkylating agent, a platinum agent, an antimetabolite, a topoisomerase inhibitor, a microtubule-targeting agent, and a cytotoxic agent.

11. The exosome composition of claim 10, wherein (i) the alkylating agent is selected from the group consisting of cyclophosphamide, ifosfamide, melphalan, and bendamustine; (ii) the platinum agent is selected from the group consisting of cisplatin, carboplatin, and oxaliplatin; (iii) the antimetabolite is selected from the group consisting of 5-fluorouracil (5-FU), capecitabine, methotrexate, cytarabine, gemcitabine, and pemetrexed; (iv) the topoisomerase inhibitor is selected from the group consisting of doxorubicin (DOX), epirubicin, irinotecan, topotecan, etoposide, and teniposide; (v) the microtubule-targeting agent is selected from the group consisting of paclitaxel, docetaxel, vincristine, vinblastine, vinorelbine, and cabazitaxel; and (vi) the cytotoxic agent is selected from the group consisting of bleomycin, dacarbazine, and temozolomide.

12. The exosome composition of claim 2, wherein the exosome composition is at least partially internalized by the cancer cell.

13. The exosome composition of claim 2, wherein the portion of the cancer cell or the tumor microenvironment comprises a cell surface protein.

14. The exosome composition of claim 13, wherein the cell surface protein is a transmembrane protein.

15. The exosome composition of claim 14, wherein the transmembrane protein is an integrin or a subunit thereof.

16. The exosome composition of claim 15, wherein the integrin is selected from the group consisting of an arginine-glycine-aspartic acid (RGD)-binding integrin, a collagen-binding integrin, a laminin-binding integrin, and a leukocyte-specific integrin.

17. The exosome composition of claim 2, wherein the one or more TSPs comprise an RGD motif.

18. The exosome composition of claim 17, wherein the one or more TSPs comprise or consist of an amino acid sequence at least about 100%, 99%, 98%, 95%, 90%, 85%, 80%, 75%, 70%, 65%, 60%, or 50% identical to the amino acid sequence of CRGDKGPDC (SEQ ID NO: 1).

19. The exosome composition of claim 2, wherein the exosome is at least partially conjugated to the one or more TSPs by a lipid-polymer conjugate.

20. The exosome composition of claim 19, wherein the lipid-polymer conjugate comprises or consists of one or more of a phospholipid, a polymer, and a functional group.

21. The exosome composition of claim 20, wherein (i) the phospholipid is 1,2-distearoyl-sn-glycero-3-phosphoethanolamine (DSPE); (ii) the polymer is poly(ethylene glycol) (PEG5000); and/or (iii) the functional group is an azide functional group.

22. The exosome composition of claim 2, wherein the exosome is selected from the group consisting of a mesenchymal stem cell-derived exosome (MSc-exosome), an adipose-derived stem cell exosome (asc-exosome), a bone marrow-derived mesenchymal stromal/stem cell exosome (bmsc-exosome), an umbilical cord mesenchymal stromal/stem cell exosome (ucmsc-exosome), a dendritic cell-derived exosome (dc-exosome), a T cell-derived exosome, a natural killer cell-derived exosome (NK cell-derived exosome), a B cell-derived exosome, a macrophage-derived exosome, an M1 macrophage-derived exosome (M1-exosome), an M2 macrophage-derived exosome (m2-exosome), a platelet-derived exosome, an erythrocyte-derived exosome, an endothelial cell-derived exosome, a fibroblast-derived exosome, a neural stem cell-derived exosome, a neuron-derived exosome, an astrocyte-derived exosome, a microglia-derived exosome, a cardiomyocyte-derived exosome, a hepatocyte-derived exosome, a tumor-derived exosome (tdex), a cancer-associated fibroblast-derived exosome (caf-derived exosome), a serum-derived exosome, a plasma-derived exosome, a urine-derived exosome, a saliva-derived exosome, a breast milk-derived exosome, an ascites-derived exosome, and a cerebrospinal fluid-derived exosome (csf-derived exosome).

23. The exosome composition of claim 2, wherein the exosome comprises (i) a particle size of about 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, or 200 nm; (ii) a zeta potential of about −100 mV, −90 mV, −80 mV, −75 mV, −70 mV, −60 mV, −65 mV, −50 mV, −45 mV, −40 mV, −35 mV, −30 mV, −20 mV, or −15 mV; (iii) an exosome:TSP weight ratio of at least about 1:2500, 1:3000, 1:4000, 1:5000, 1:5500, 1:6000, 1:6500, 1:7000, 1:8000, 1:9000, or 1:10000; and (iv) a weight ratio of TSP and exosomes:anticancer agent of about 10:1, 11:1, 12:1, 13:1, 14:1, 15:1, 16:1, 17:1, 18:1, 19:1, or 20:1.

24. The exosome composition of claim 2, wherein the one or more anticancer agents comprise or consist of two or more anticancer agents.

25. The exosome composition of claim 2, wherein the one or more anticancer agents are present in the exosome in an amount of about 10 ng/ml, 20 ng/ml, 50 ng/ml, 100 ng/ml, 200 ng/ml, 500 ng/ml, 1 μg/mL, 2 μg/mL, 5 μg/mL, 10 μg/mL, 20 μg/mL, 50 μg/mL, 100 μg/mL, 200 μg/mL, 500 μg/mL, 1 mg/mL, 1.5 mg/mL, 2 mg/ml, 3 mg/mL, 5 mg/mL, 10 mg/mL, 15 mg/mL, or 20 mg/mL.

26. The method of claim 3, wherein the subject has or is at risk of (i) developing TNBC or (ii) developing primary breast cancer or metastatic breast cancer.

27. The exosome composition of claim 2, wherein the exosome composition is formulated in a pharmaceutical composition.

28. The exosome composition of claim 27, wherein the pharmaceutical composition further comprises one or more pharmaceutically acceptable carriers or excipients.

29. The exosome composition of claim 28, wherein the pharmaceutical composition is formulated for oral, rectal, vaginal, ocular, intranasal, topical, parenteral, injectable delivery, or from a surgical device.

30. The method of claim 3, wherein the exosome composition is administered to the subject in an amount of about (i) 1.5 mg/kg/day to about 45 mg/kg/day of the exosome composition or (ii) 0.01 mg/kg/day to about 10.0 mg/kg/day, of the one or more anticancer agents.
